(19) United States
(12) Patent Application Publication
(10) Pub. No.: US 2018/0247770 A1
(43) Pub. Date: Aug. 30, 2018

(54) HETEROCYCLIC COMPOUND FOR ORGANIC ELECTRONIC DEVICE AND USING THE SAME

(71) Applicants: FENG-WEN YEN, Taipei (TW); Chin-Min Teng, Miaoli (TW)

(72) Inventors: FENG-WEN YEN, Taipei (TW); Chin-Min Teng, Miaoli (TW)

(73) Assignee: LUMINESCENCE TECHNOLOGY CORPORATION, Hsinchu (TW)

(21) Appl. No.: 15/442,713

(22) Filed: Feb. 27, 2017

(51) Int. Cl. H01G 9/20; C07D 519/00; H01L 51/00; C07D 495/22

(52) U.S. Cl. CPC H01G 9/2018; C07D 519/00; H01L 51/0074; C07D 495/22; H01L 51/0558; H01L 51/0067; H01L 51/0077; H01L 51/0037; H01L 51/0035; H01L 51/0061

(57) ABSTRACT

The present invention discloses a heterocyclic compound represented by the following formula(I), and an organic electronic device using the heterocyclic compound can display good performance. Especially, the heterocyclic compound is suitable for organic semiconductor device, pervoskite solar cell device, and organic electroluminescence (organic EL) device.

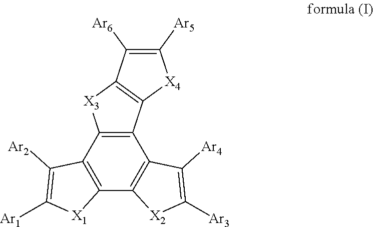

wherein X₁ to X₄ each independently represent a sulfur atom or a selenium atom, Ar₁ to Ar₆ are the same definition as described in the present invention.

### FIELD OF INVENTION

The present invention generally relates to a heterocyclic compound and organic electronic device using the heterocyclic compound, More specifically, the heterocyclic compound is suitable for an organic semiconductor device, a pervoskite solar cell device, and organic electroluminescence (organic EL) device. Additional, the present invention employs the heterocyclic compound as hole transport layer (HTL) or electron transport layer (ETL) materials for pervoskite solar cell device and organic EL device can display excellent performance.

### BACKGROUND OF THE INVENTION

Organic electronic material has been developed for several decades. Recently the organic electronic material are widely put in use in organic electronic devices, such as OTFT, organic EL device, OPV device, and pervoskite solar cell device have attracted significant attention for industries practice use due to their potential application for flat-panel and flexible display, solid-state lighting, solar energy storage, etc. Organic EL is a light-emitting diode (LED) in which the emissive layer is a film made by organic compounds which emits light in response to an electric current. The emissive layer of organic compound is sandwiched between two electrodes. Organic EL device have many advantages such as self-emitting, wider viewing angles, faster response speeds and highly luminescence. Their simpler fabrication and capable of giving clear display comparable with LCD, making organic EL device an industry display of choice and has stepped into commercialization. An organic photovoltaic (OPV) device includes a substrate, a first electrode, a second electrode and a photoelectric conversion layer. The first electrode is disposed on the substrate. The second electrode is disposed on the first electrode. The photoelectric conversion layer is disposed between the first electrode and the second electrode. The device cell has electrical charge generated by absorbing the light. OPV has been considered as a highly growing trend for green energy technology because of its low cost, simple preparation and large area capability. The conversion efficiency of OPV had reached to the practical application. An organic thin-film transistor (OTFT) including, on a substrate having an insulating surface, at least a gate electrode, a gate insulating film formed in contact with the gate electrode, an organic semiconductor film formed in contact with the gate insulating film, and at least a pair of source-drain electrodes formed in contact with the organic semiconductor film, a carrier generating electrode to which carriers can be injected in response to a gate signal is implanted within the organic semiconductor film. OTFT has grown into a hotspot in organic electronics as it also possesses the merits of low cost, flexibility, low temperature processing and large area capability. And its performance is already comparable to that of the amorphous silicon based thin film transistors.

Recently, the importance of a solar cell is ever-increasing as an alternative energy to fossil fuel. However, the cost of present solar cells as typified by a silicon-based solar cell is high. Thus, various inexpensive solar cells are in research and development, which a dye sensitization type solar cell announced by Graetzel et al. of Ecole Polytechnique Federale de Lausanne is highly anticipated (disclosed in JP Patent No. 2664194; Nature, 353(1991) 737; and J. Am. Chem. Soc., 115(1993) 6382).

A perovskite solar cell in which a perovskite structure compound absorbs light and generates electric power was announced by Miyasaka et al. of Toin University of Yokohama in J. Am. Chem. Soc., 131(2009) 6050. The perovskite structure compound employed in the perovskite solar cell is formed by mixing halogenated methylamine and lead halide. The perovskite structures compound exhibits strong absorption with respect to visible light. A perovskite solar cell in which photoelectric conversion efficiency was enhanced was announced in Science 338(2012) 643. The perovskite solar cell announced in Science 338(2012) 643, it cannot be said to obtain photoelectric conversion efficiency that is sufficiently satisfactory. Thus, there is a demand for higher photoelectric conversion efficiency.

The performance of organic semiconductor devices is principally based upon the charge carrier mobility of the semiconducting material and the current on/off ratio, so the ideal semiconductor should have a low conductivity in the off state, combined with a high charge carrier mobility (>1×10⁻³ cm² V⁻¹ s⁻¹). In addition, it is important that the semiconducting material is relatively stable to oxidation i.e. it has a high ionization potential, as oxidation leads to reduced device performance. Further requirements for the semiconducting material are good process ability, especially for large-scale production of thin layers and desired patterns, and high stability, film uniformity and integrity of the organic semiconductor layer.

However, there are still many technical problems remaining to be solved in organic electronic devices, such as material instability, low power efficiency, short life time, etc., which hindered the commercialization of organic electronic devices.

There continues to be a need for organic electronic materials which having good thermal stability and more efficient and long half-life time for organic electronic device.

The prior art of the novel heterocyclic compound for the present invention including U.S. Pat. No. 8,313,672B2, JP 2005-156822A1, Org. Lett., Vol. 6, No. 2, 273-276 (2004), Inorg. Chem. 2011, 50, 471-478.

### SUMMARY OF THE INVENTION

Provided a novel heterocyclic compound as hole transport layer (HTL), electron transport layer (ETL) or active layer for organic electronic devices (organic EL, OPV, pervoskite solar cell or OTFT), the heterocyclic compound can overcome the drawbacks of the conventional materials like as lower stability, lower half-lifetime and higher power consumption.

The present invention has the economic advantages for industrial practice. Accordingly the present invention, a heterocyclic compound represented by the formula (I) as the follows, the heterocyclic compound is suitable for an organic semiconductor device, a pervoskite solar cell device, and organic electroluminescence (organic EL) device. Additional, the present invention employs the heterocyclic compound as hole transport layer (HTL) or electron transport layer (ETL) materials for pervoskite solar cell device and organic EL device can display excellent performance.

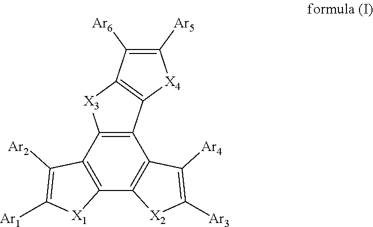

wherein X₁ to X₄ each independently represent a sulfur or a selenium atom, Ar₁ to Ar₆ are identical or different, Ar₁ to Ar₆ are independently selected from the group consisting of a hydrogen atom, a halide, —CN, —NC, —NCS, —SCN, —NH₂, —OH, —NO₂, —CF₃, —NC, a substituted or unsubstituted alkyl group having 1 to 30 carbon atoms, a substituted or unsubstituted arvl group having 6 to 30 carbon atoms, a substituted or unsubstituted heteroaryl group having 3 to 30 carbon atoms, a substituted or unsubstituted aralkyl group having 6 to 30 carbon atoms, a substituted or unsubstituted alkoxyl group having 6 to 30 carbon atoms, a substituted or unsubstituted alkenyl group having 6 to 30 carbon atoms and a substituted or unsubstituted alkylamine group having 6 to 30 carbon atoms.

### BRIEF DESCRIPTION OF THE DRAWINGS

FIG. 1 show the drawing of OTFT device in the present invention.

FIG. 2 show the drawing of pervoskite solar cell device in the present invention.

### DESCRIPTION OF THE PREFERRED EMBODIMENTS

What probed into the invention is the heterocyclic compound and organic electronic device using the heterocyclic compound. Detailed descriptions of the production, structure and elements will be provided in the following to make the invention thoroughly understood. Obviously, the application of the invention is not confined to specific details familiar to those who are skilled in the art. On the other hand, the common elements and procedures that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In a first embodiment of the present invention, the organic electronic material which can be used for organic EL device, OPV device pervoskite solar cell device or OTFT device are disclosed. The mentioned organic electronic materials are represented by the following formula(1):

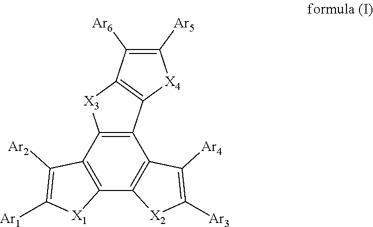

wherein X₁ to X₄ each independently represent a sulfur or a selenium atom, Ar₁ to Ar₆ are identical or different, Ar₁ to Ar₆ are independently selected from the group consisting of a hydrogen atom, a halide, —CN, —NC, —NCS, —SCN, —NH₂, —OH, —NO₂, —CF₃, —NC, a substituted or unsubstituted alkyl group having 1 to 30 carbon atoms, a substituted or unsubstituted aryl group having 6 to 30 carbon atoms, a substituted or unsubstituted heteroaryl group having 3 to 30 carbon atoms, a substituted or unsubstituted aralkyl group having 6 to 30 carbon atoms, a substituted or unsubstituted alkoxyl group having 6 to 30 carbon atoms, a substituted or unsubstituted alkenyl group having 6 to 30 carbon atoms and a substituted or unsubstituted alkylamine group having 6 to 30 carbon atoms.

According to the above-mentioned formula(I), the Ar₁ to Ar₆ group represented as the follows:

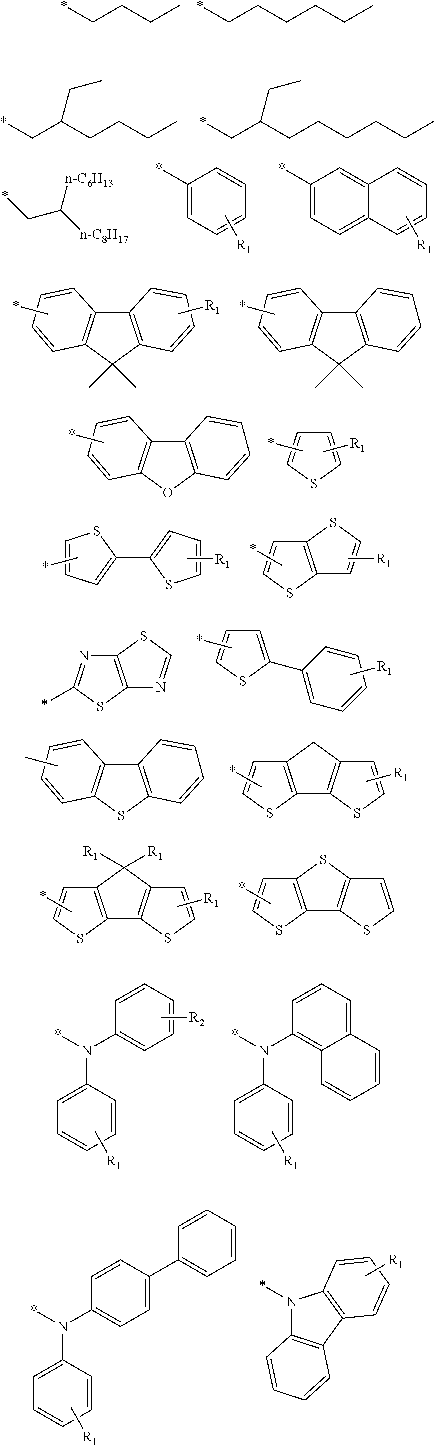

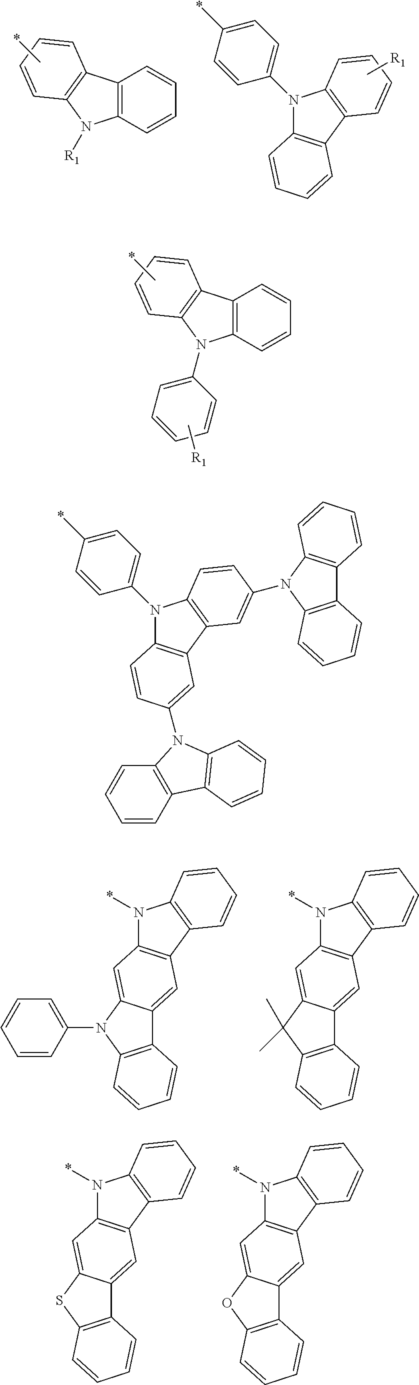

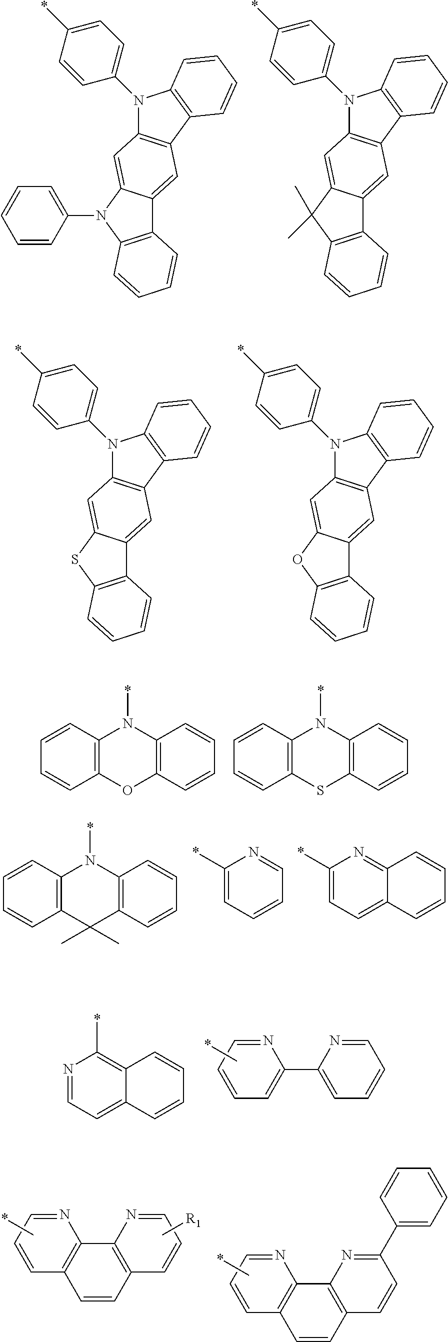

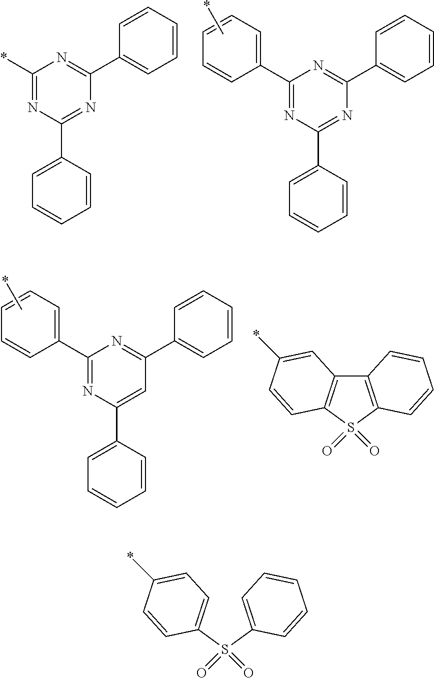

wherein R₁ and R₂ represent alkyl group, or aryl group.

In this embodiment, heterocyclic compounds are shown below:

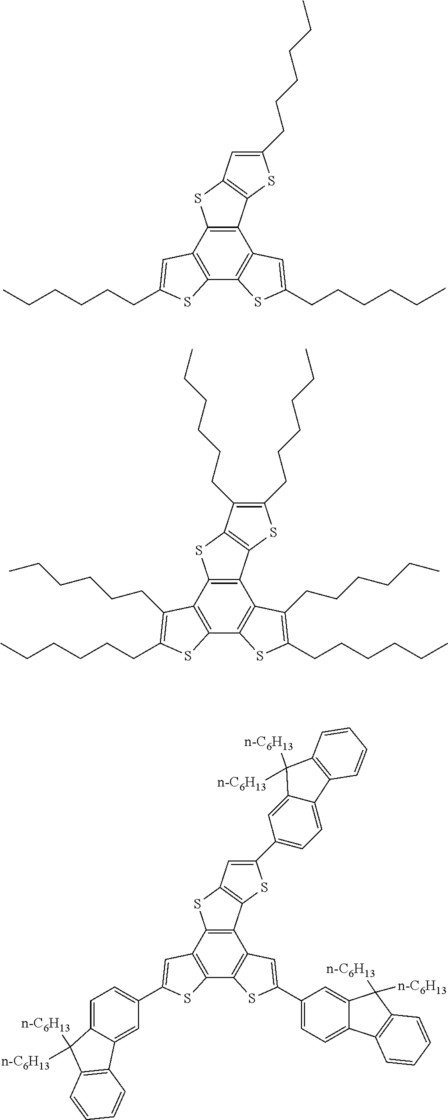

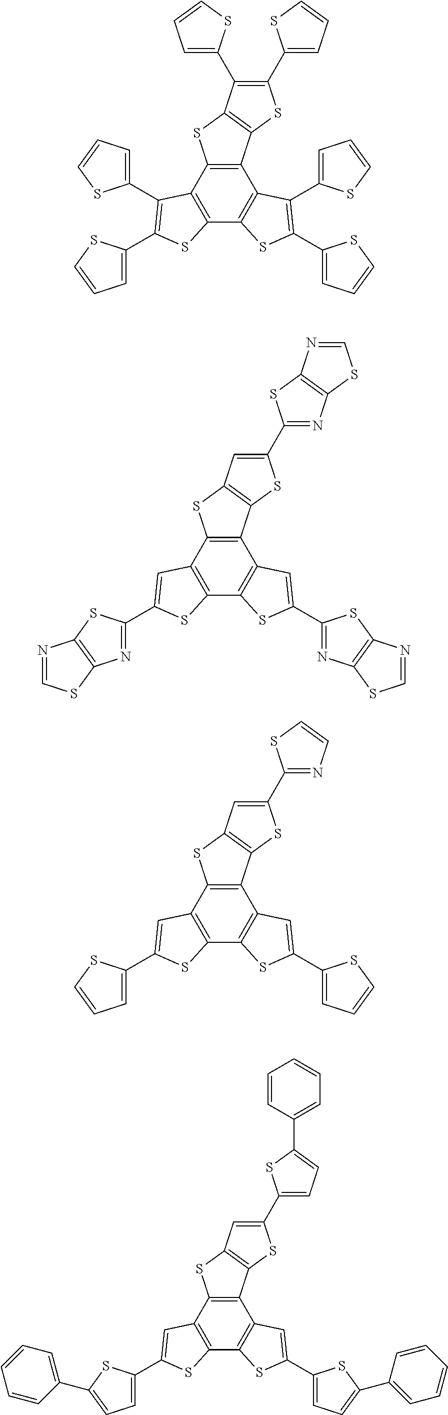 [SMILES: c1csc(-c2sc3c(sc4c5c(-c6cccs6)c(-c6cccs6)sc5c5sc(-c6cccs6)c(-c6cccs6)c5c34)c2-c2cccs2)c1 ; c1nc2sc(-c3cc4c(s3)c3sc(-c5nc6scnc6s5)cc3c3c5sc(-c6nc7scnc7s6)cc5sc43)nc2s1 ; c1csc(-c2cc3c(s2)c2sc(-c4cccs4)cc2c2c4sc(-c5nccs5)cc4sc32)c1 ; c1ccc(-c2ccc(-c3cc4c(s3)c3sc(-c5ccc(-c6ccccc6)s5)cc3c3c5sc(-c6ccc(-c7ccccc7)s6)cc5sc43)s2)cc1]

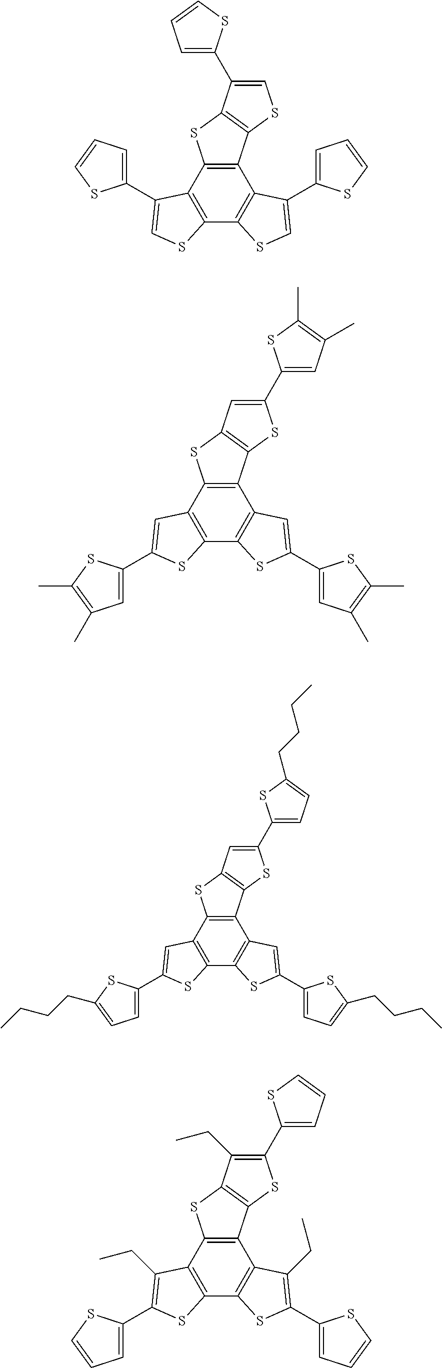

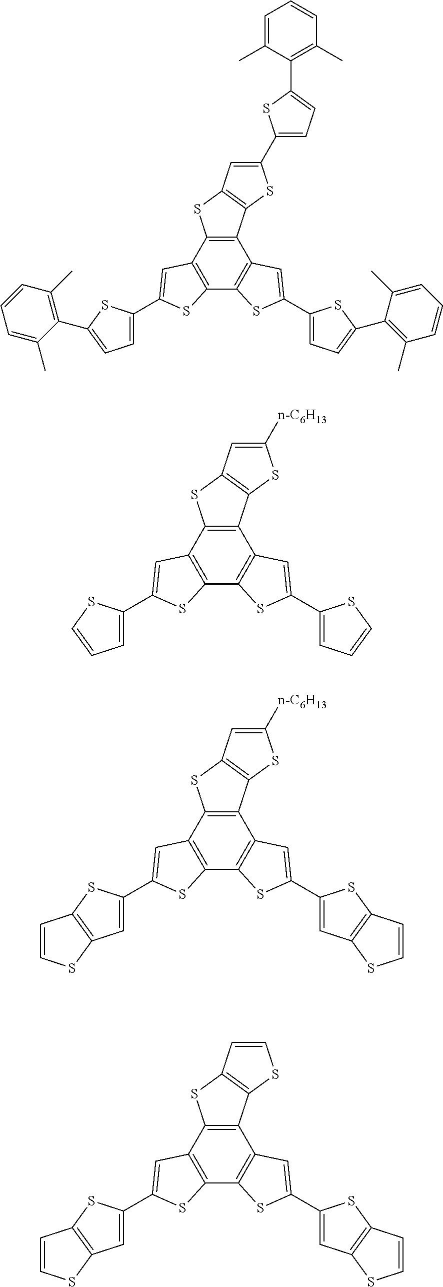

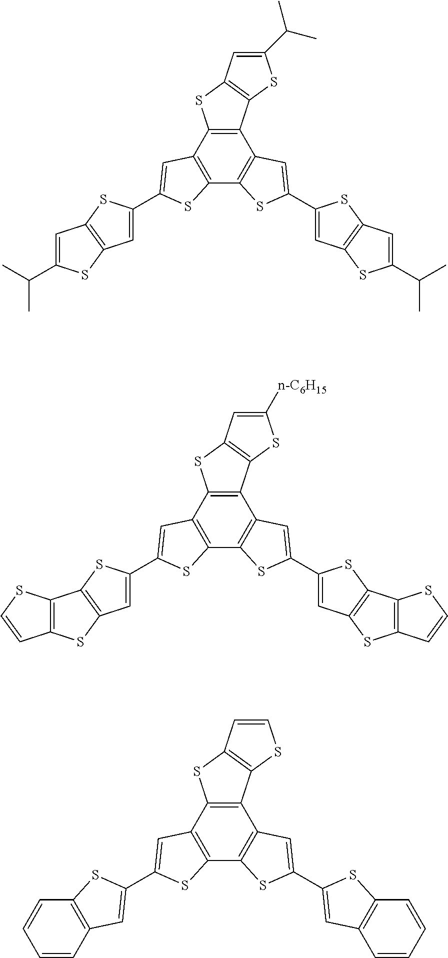

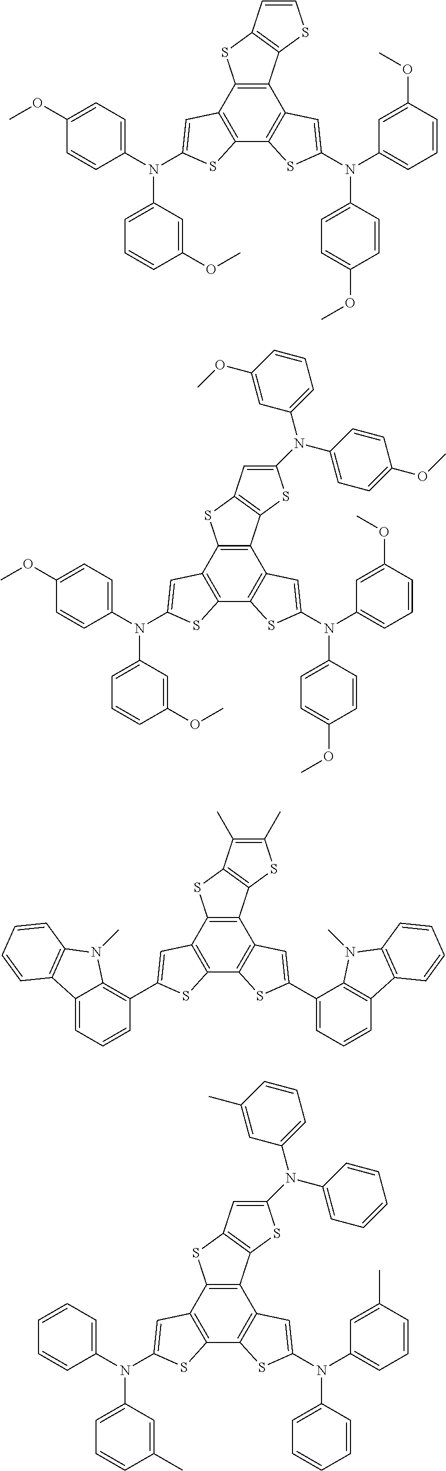

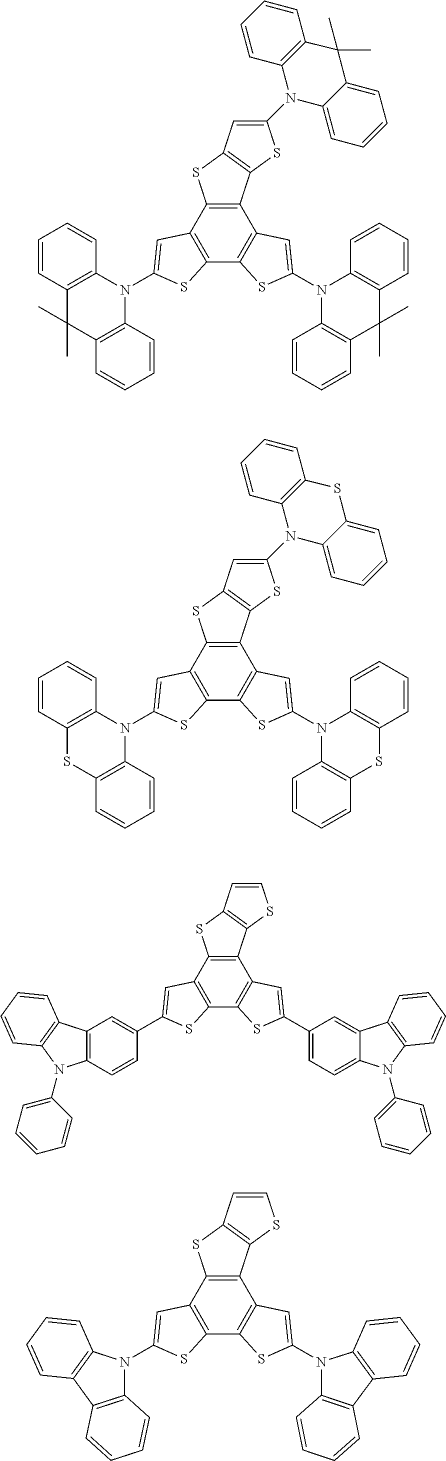 [SMILES: CC1(C)c2ccccc2N(c2cc3c(s2)c2sc(N4c5ccccc5C(C)(C)c5ccccc54)cc2c2c4sc(N5c6ccccc6C(C)(C)c6ccccc65)cc4sc32)c2ccccc21 ; c1ccc2c(c1)Sc1ccccc1N2c1cc2c(s1)c1sc(N3c4ccccc4Sc4ccccc43)cc1c1c3sc(N4c5ccccc5Sc5ccccc54)cc3sc21 ; c1ccc(-n2c3ccccc3c3cc(-c4cc5c(s4)c4sc(-c6ccc7c(c6)c6ccccc6n7-c6ccccc6)cc4c4c6sccc6sc54)ccc32)cc1 ; c1ccc2c(c1)c1ccccc1n2-c1cc2c(s1)c1sc(-n3c4ccccc4c4ccccc43)cc1c1c3sccc3sc21]

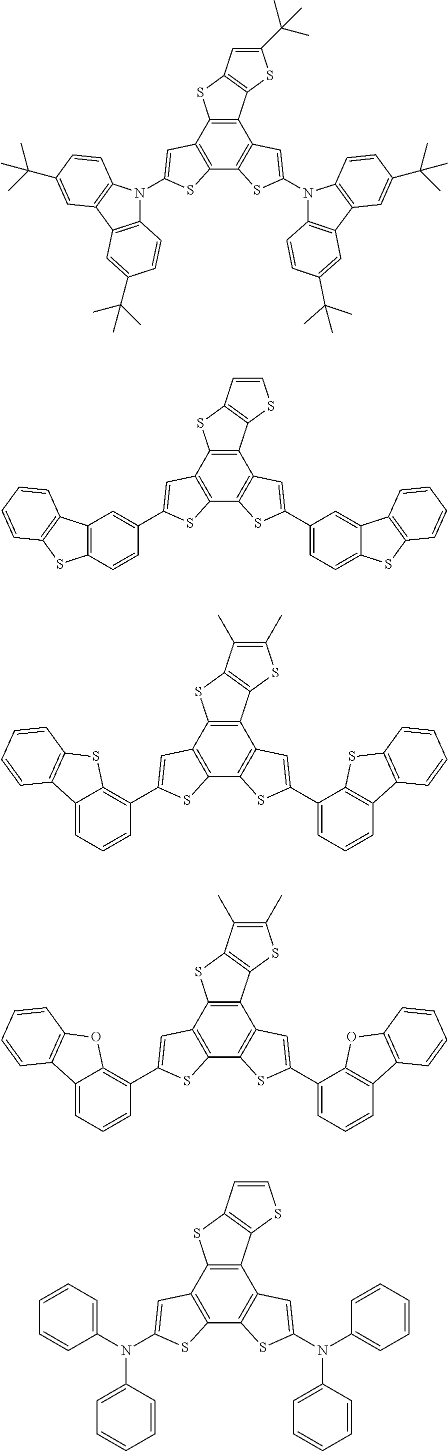 [SMILES: CC(C)(C)c1ccc2c(c1)c1cc(C(C)(C)C)ccc1n2-c1cc2c(s1)c1sc(-n3c4ccc(C(C)(C)C)cc4c4cc(C(C)(C)C)ccc43)cc1c1c3sc(C(C)(C)C)cc3sc21 ; c1ccc2c(c1)sc1ccc(-c3cc4c(s3)c3sc(-c5ccc6sc7ccccc7c6c5)cc3c3c5sccc5sc43)cc12 ; Cc1sc2c(sc3c4cc(-c5cccc6c5sc5ccccc56)sc4c4sc(-c5cccc6c5sc5ccccc56)cc4c23)c1C ; Cc1sc2c(sc3c4cc(-c5cccc6c5oc5ccccc56)sc4c4sc(-c5cccc6c5oc5ccccc56)cc4c23)c1C ; c1ccc(N(c2ccccc2)c2cc3c(s2)c2sc(N(c4ccccc4)c4ccccc4)cc2c2c4sccc4sc32)cc1]

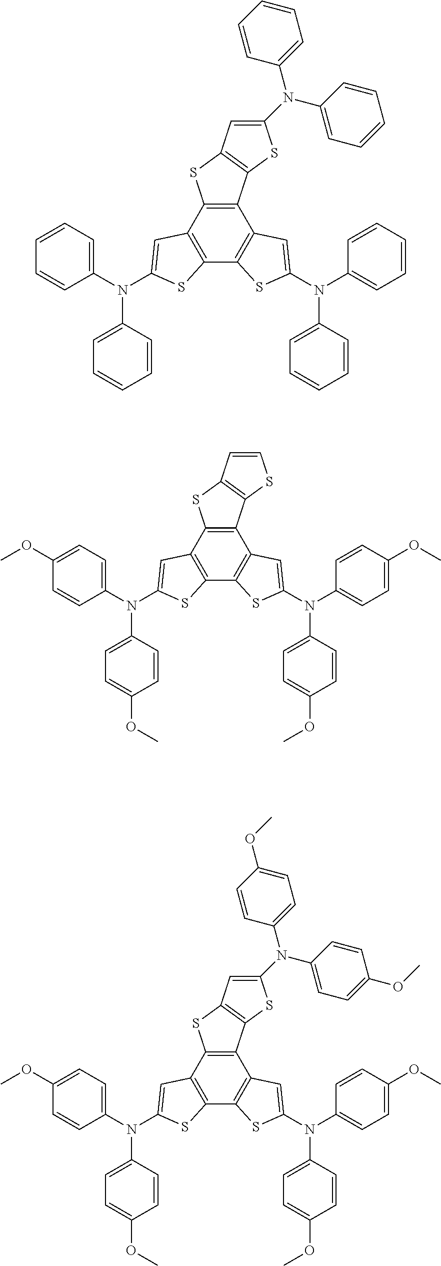

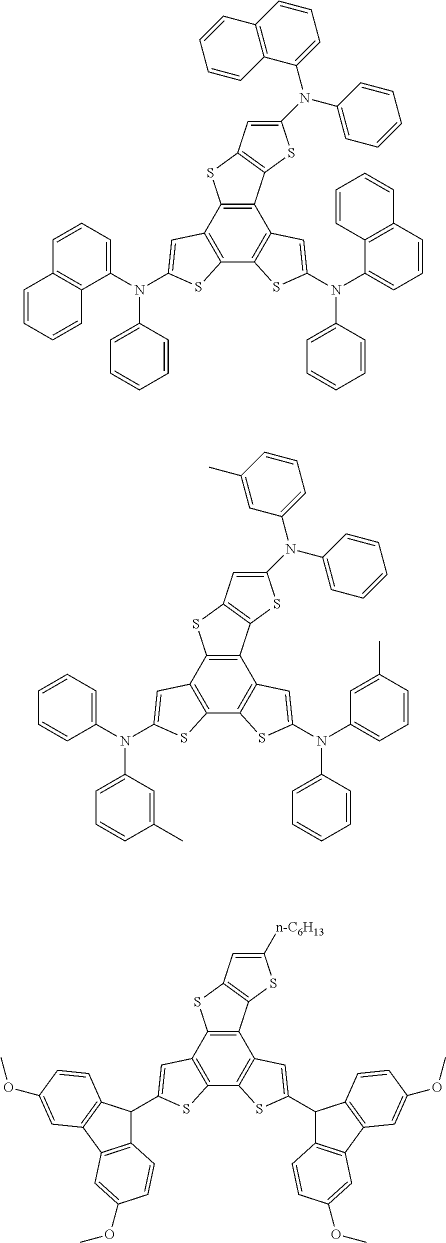

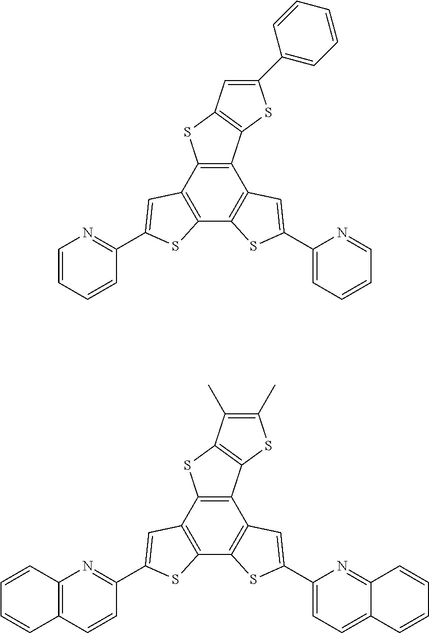 [SMILES: c1ccc(-c2cc3sc4c5cc(-c6ccccn6)sc5c5sc(-c6ccccn6)cc5c4c3s2)cc1 ; Cc1sc2c(sc3c4cc(-c5ccc6ccccc6n5)sc4c4sc(-c5ccc6ccccc6n5)cc4c23)c1C]

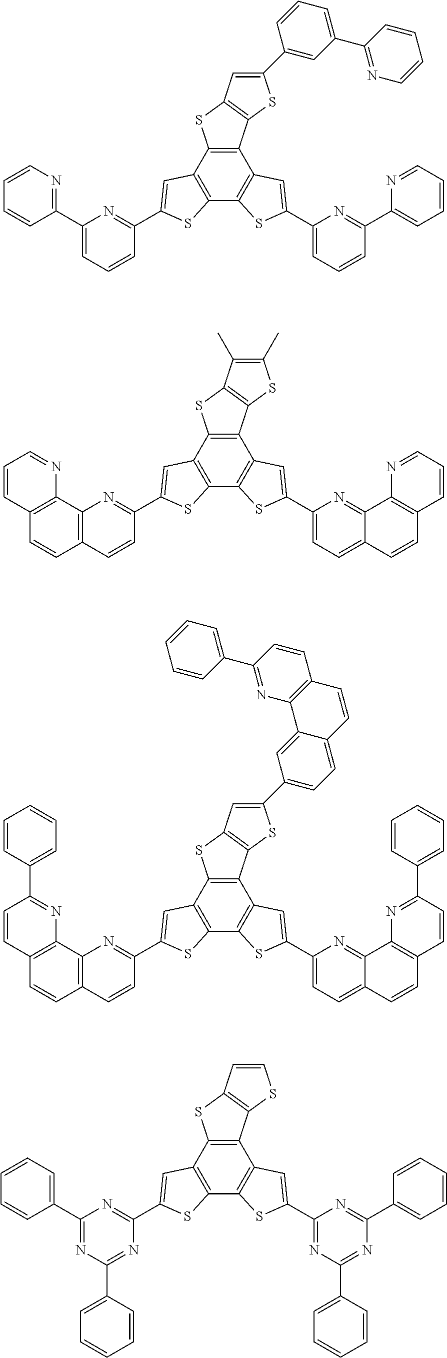 [SMILES: c1ccc(-c2cccc(-c3cc4sc5c6cc(-c7cccc(-c8ccccn8)n7)sc6c6sc(-c7cccc(-c8ccccn8)n7)cc6c5c4s3)c2)nc1 ; Cc1sc2c(sc3c4cc(-c5ccc6ccc7cccnc7c6n5)sc4c4sc(-c5ccc6ccc7cccnc7c6n5)cc4c23)c1C ; c1ccc(-c2ccc3ccc4ccc(-c5cc6sc7c8cc(-c9ccc%10ccc%11ccc(-c%12ccccc%12)nc%11c%10n9)sc8c8sc(-c9ccc%10ccc%11ccc(-c%12ccccc%12)nc%11c%10n9)cc8c7c6s5)cc4c3n2)cc1 ; c1ccc(-c2nc(-c3ccccc3)nc(-c3cc4c(s3)c3sc(-c5nc(-c6ccccc6)nc(-c6ccccc6)n5)cc3c3c5sccc5sc43)n2)cc1]

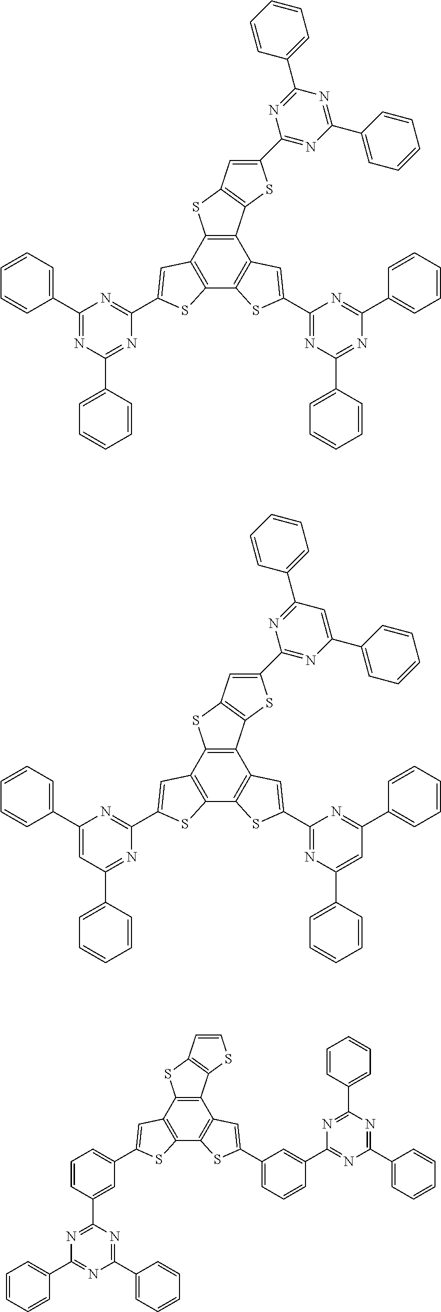 [SMILES: c1ccc(-c2nc(-c3ccccc3)nc(-c3cc4c(s3)c3sc(-c5nc(-c6ccccc6)nc(-c6ccccc6)n5)cc3c3c5sc(-c6nc(-c7ccccc7)nc(-c7ccccc7)n6)cc5sc43)n2)cc1 ; c1ccc(-c2cc(-c3ccccc3)nc(-c3cc4c(s3)c3sc(-c5nc(-c6ccccc6)cc(-c6ccccc6)n5)cc3c3c5sc(-c6nc(-c7ccccc7)cc(-c7ccccc7)n6)cc5sc43)n2)cc1 ; c1ccc(-c2nc(-c3ccccc3)nc(-c3cccc(-c4cc5c(s4)c4sc(-c6cccc(-c7nc(-c8ccccc8)nc(-c8ccccc8)n7)c6)cc4c4c6sccc6sc54)c3)n2)cc1]

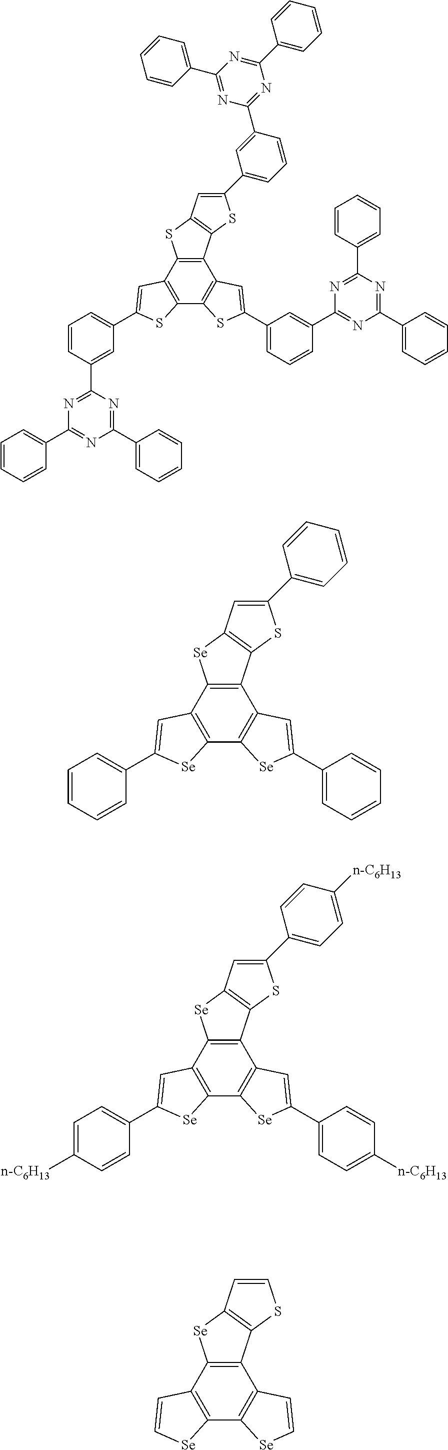

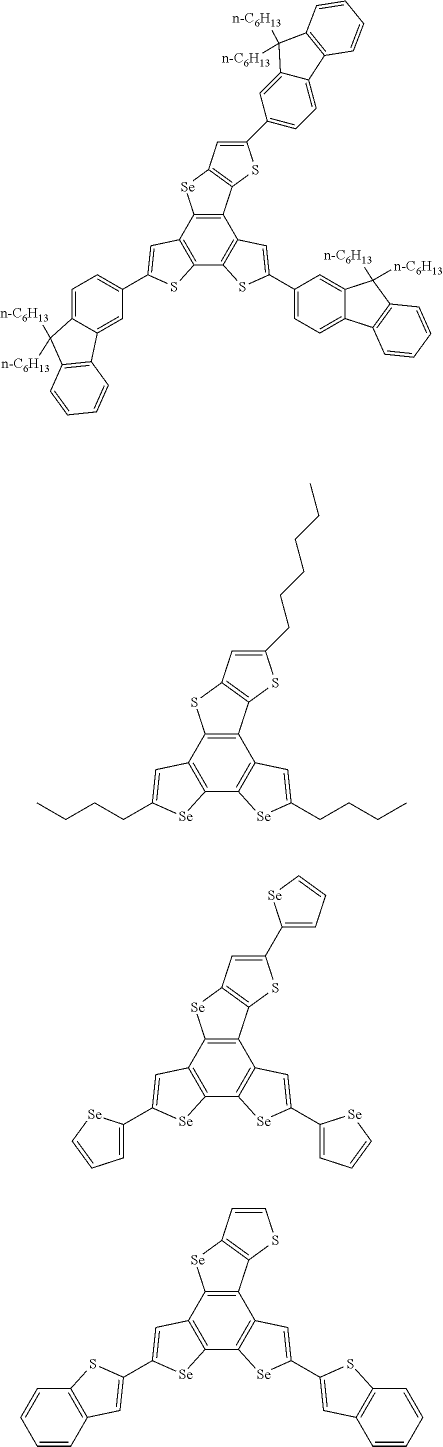

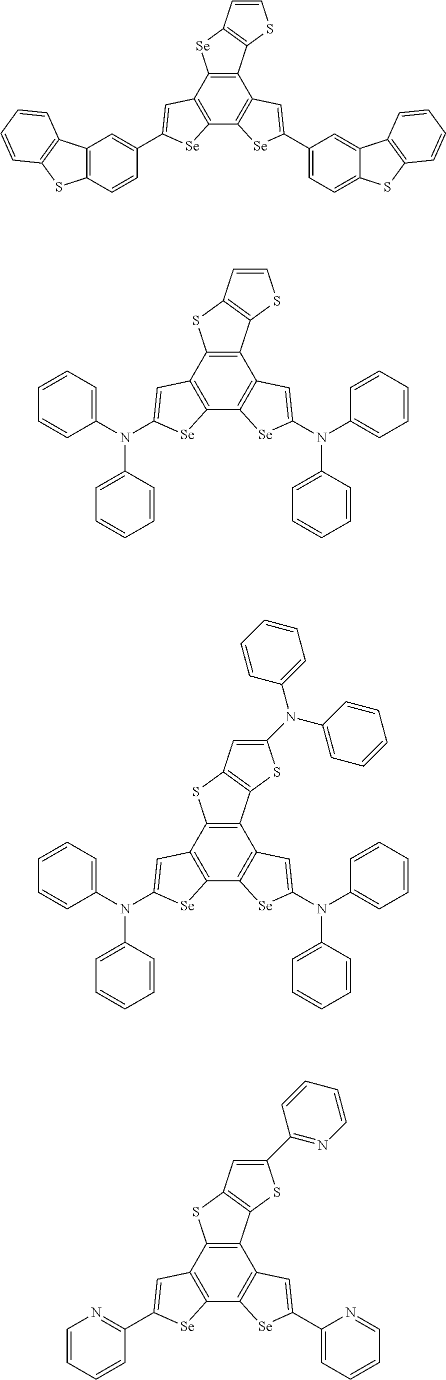 [SMILES: c1ccc2c(c1)sc1ccc(-c3cc4c([se]3)c3[se]c(-c5ccc6sc7ccccc7c6c5)cc3c3c5sccc5[se]c43)cc12 ; c1ccc(N(c2ccccc2)c2cc3c4sc5ccsc5c4c4cc(N(c5ccccc5)c5ccccc5)[se]c4c3[se]2)cc1 ; c1ccc(N(c2ccccc2)c2cc3sc4c5cc(N(c6ccccc6)c6ccccc6)[se]c5c5[se]c(N(c6ccccc6)c6ccccc6)cc5c4c3s2)cc1 ; c1ccc(-c2cc3sc4c5cc(-c6ccccn6)[se]c5c5[se]c(-c6ccccn6)cc5c4c3s2)nc1]

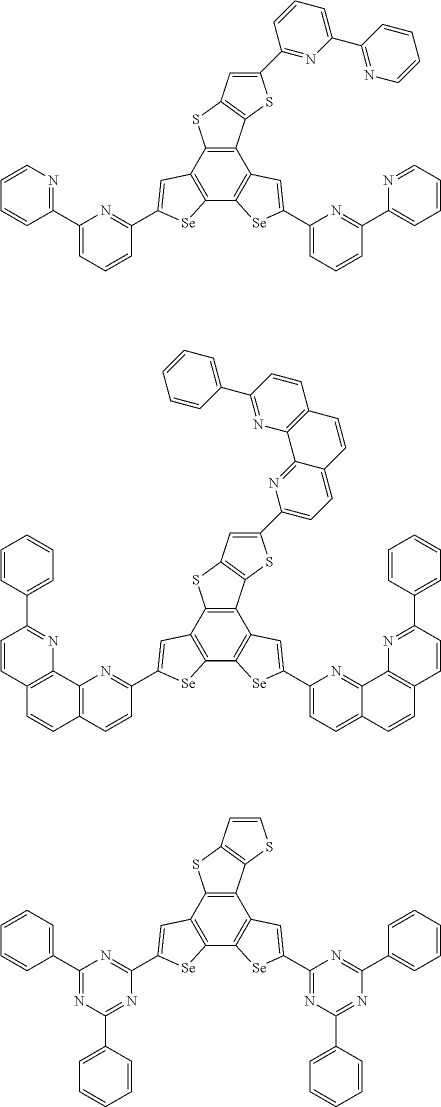 [SMILES: c1ccc(-c2cccc(-c3cc4sc5c6cc(-c7cccc(-c8ccccn8)n7)[se]c6c6[se]c(-c7cccc(-c8ccccn8)n7)cc6c5c4s3)n2)nc1 ; c1ccc(-c2ccc3ccc4ccc(-c5cc6sc7c8cc(-c9ccc%10ccc%11ccc(-c%12ccccc%12)nc%11c%10n9)[se]c8c8[se]c(-c9ccc%10ccc%11ccc(-c%12ccccc%12)nc%11c%10n9)cc8c7c6s5)nc4c3n2)cc1 ; c1ccc(-c2nc(-c3ccccc3)nc(-c3cc4c5sc6ccsc6c5c5cc(-c6nc(-c7ccccc7)nc(-c7ccccc7)n6)[se]c5c4[se]3)n2)cc1]

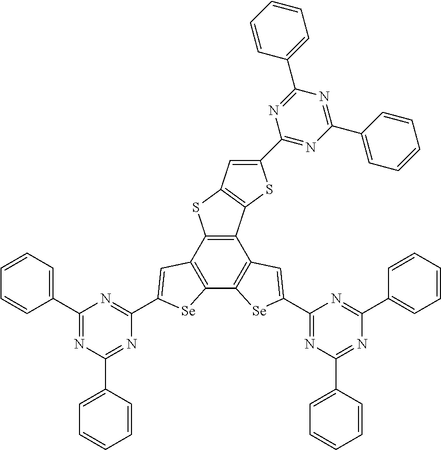 [SMILES: c1ccc(-c2nc(-c3ccccc3)nc(-c3cc4sc5c6cc(-c7nc(-c8ccccc8)nc(-c8ccccc8)n7)[se]c6c6[se]c(-c7nc(-c8ccccc8)nc(-c8ccccc8)n7)cc6c5c4s3)n2)cc1]

Detailed preparation for the compound in the present invention could be clarified by exemplary embodiments, but the present invention is not limited to exemplary embodiments. EXAMPLE 1˜6 show the preparation for some EXAMPLES of the compound in the present invention. EXAMPLE 7 shows the fabrication of organic TFT device and I-V-B, half-life time of organic EL device testing report. EXAMPLE 8 shows the fabrication of pervoskite solar cell device and I-V & PCE testing report.

### Example 1

Synthesis of Compound 1

Synthesis of Intermediate A

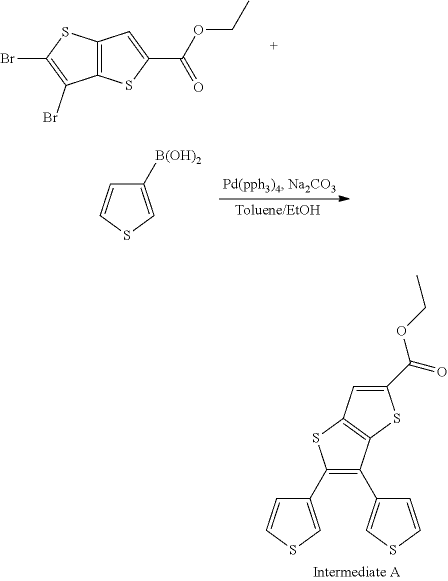 [SMILES: CCOC(=O)c1cc2sc(Br)c(Br)c2s1 ; OB(O)c1ccsc1 ; CCOC(=O)c1cc2sc(-c3ccsc3)c(-c3ccsc3)c2s1]

A mixture of 25 g (67.6 mmol) of ethyl 5,6-dibromothieno[3,2-b]thiophene-2-carboxylate (the compound was synthesized as Inorg. Chem. 2011, 50, 471-478), 13.0 g (101.3 mmol) of thiophen-3-ylboronic acid, 0.8 g (0.067 mmol) of Pd(PPh₃)₄, 101 ml of 2M Na₂CO₃, 300 ml of toluene and 100 ml EtOH was degassed and placed under nitrogen, and then heated at 100° C. for 12 h. After finishing the reaction, the mixture was allowed to cool to room temperature. The organic layer was extracted with dichloromethane and water, dried with anhydrous magnesium sulfate, the solvent was removed and the residue was purified by column chromatography on silica to give product (16.8 g, 66%).

Synthesis of Intermediate B

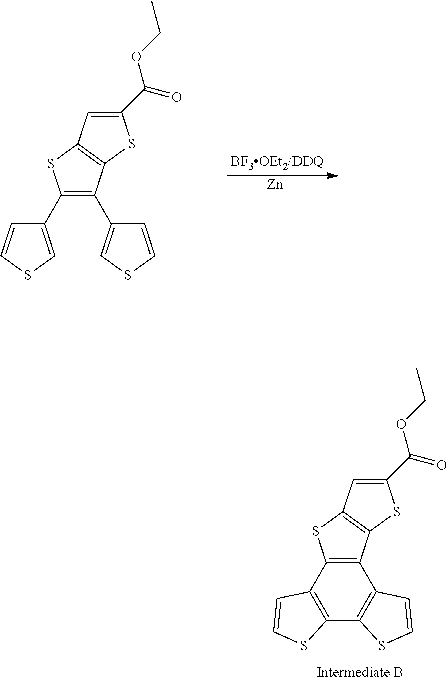

In a three-necked flask that had been degassed and filled with nitrogen, a mixture of 1.6 g (4.4 mmol) of Intermediate A, 0.95 g (6.7 mmol) of boron trifluoride diethyl etherate and 1.6 g (7.0 mmol) of D.D.Q. was dissolved in anhydrous dichloromethane (425 ml), and the mixture was stirred at room temperature for 24 h. A mixture of 0.03 g (0.44 mmol) Zinc and 850 ml of MeOH was then added, and the mixture was stirred at room temperature for 24 h. Water and dichloromethane was added to the mixture for quenched. And the organic layer was separated and the solvent removed in vacuo. The residue was purified by column chromatography on silica to give product (0.7 g, 42%).

Synthesis of Intermediate C

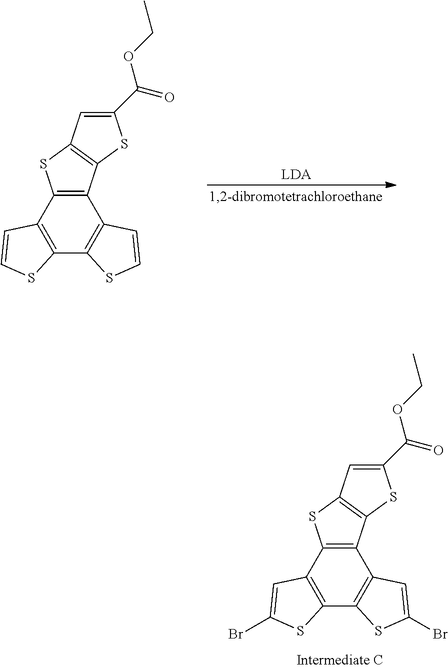 [SMILES: CCOC(=O)c1cc2sc3c4ccsc4c4sccc4c3c2s1 ; CCOC(=O)c1cc2sc3c4cc(Br)sc4c4sc(Br)cc4c3c2s1]

A solution of LDA (74.8 mmol) in THF was dropwise to a solution of 7.0 g (18.7 mmol) of Intermediate C in THF (140 mL) under a nitrogen atmosphere at −78° C. The mixture was keep the temperature for 1 h and stirred at −78° C. for 1 h, then 30.4 g (93 mmol) of 1,2-dibromotetrachloroethane was dropwise to the mixture at −78° C., then allowed to warm to room temperature and stirred overnight. Water was added to the mixture for quenched. And the organic layer was separated and the solvent removed in vacuo. The residue was purified by column chromatography on silica to give product (5.9 g, 60%).

Synthesis of Intermediate D

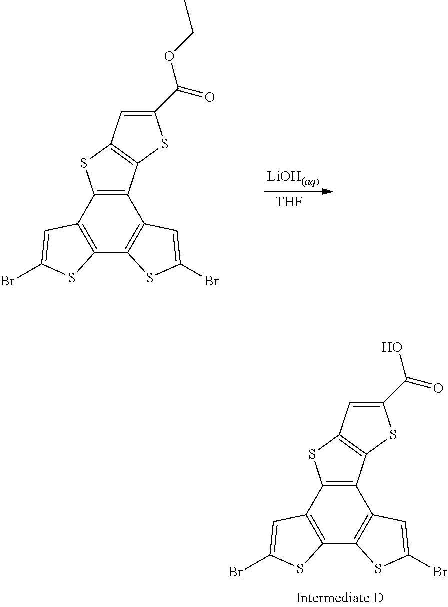 [SMILES: CCOC(=O)c1cc2sc3c4cc(Br)sc4c4sc(Br)cc4c3c2s1 ; O=C(O)c1cc2sc3c4cc(Br)sc4c4sc(Br)cc4c3c2s1 ; "THF"]

A mixture of 5.9 g (11.2 mmol) of Intermediate C, 1M LiOH_{(aq)} (22.4 mmol) and THF (60 ml). the mixture was heated at 60° C. for 2 h. After finishing the reaction, the mixture was allowed to cool to room temperature. The organic layer was extracted with ethyl acetate and water, dried with anhydrous magnesium sulfate, the solvent was removed and the residue was purified by column chromatography on silica to give product (5.3 g, 95%).

Synthesis of Intermediate E

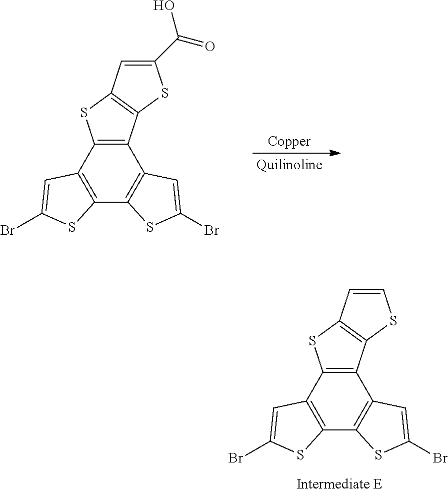 [SMILES: O=C(O)c1cc2sc3c4cc(Br)sc4c4sc(Br)cc4c3c2s1 ; Brc1cc2c(s1)c1sc(Br)cc1c1c3sccc3sc21]

A mixture of 5.3 g (10.6 mmol) of Intermediate E, 0.4 g (6.3 mmol) of copper and Quinoline (40 ml) was heated at reflux and stir 2 h until the reaction finished. The reaction mixture was cooled down and extracted with dichloromethane and water, dried with anhydrous MgSO4, the solvent was removed to give crude (2.6 g, 50%).

Synthesis of Intermediate F

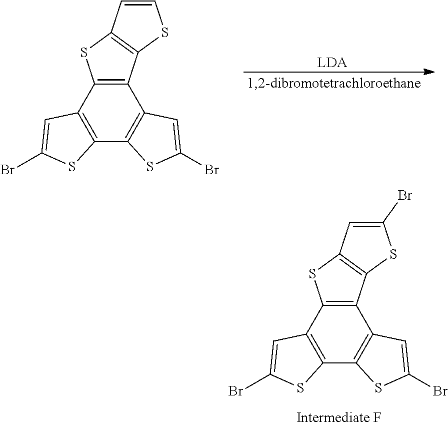 [SMILES: Brc1cc2c(s1)c1sc(Br)cc1c1c3sccc3sc21 ; Brc1cc2c(s1)c1sc(Br)cc1c1c3sc(Br)cc3sc21]

A solution of LDA (10.6 mmol) in THF was dropwise to a solution of 2.6 g (5.3 mmol) of Intermediate E in THF (50 mL) under a nitrogen atmosphere at −78° C. The mixture was keep the temperature for 1 h and stirred at −78° C. for 1 h, then 4.3 g (13.3 mmol) of 1,2-dibromotetrachloroethane was dropwise to the mixture at −78° C., then allowed to warm to room temperature and stirred overnight. Water was added to the mixture for quenched. And the organic layer was separated and the solvent removed in vacuo. The residue was purified by column chromatography on silica to give product (2 g, 72%).

Synthesis of Intermediate G

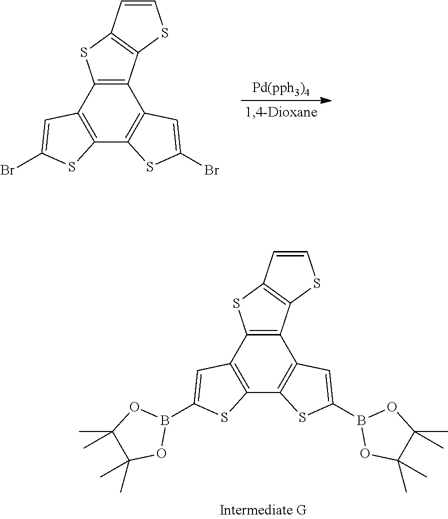

A mixture of 5 g (10.9 mmole) of Intermediate E, 6.7 g (26.2 mmol) of bis(pinacolato)diboron, 0.12 g (0.11 mmol) of Pd(PPhs)₄, 3.2 g (32.7 mmol) of potassium acetate, and 75 ml 1,4-dioxane was degassed and placed under nitrogen, and then heated at 90° C. for 16 h. After finishing the reaction, the mixture was allowed to cool to room temperature. The organic phase separated and washed with ethyl acetate and water. After drying over magnesium sulfate, the solvent was removed in vacuo. The residue was purified by column chromatography on silica to give product 4.9 g (81%).

Synthesis of Compound 1

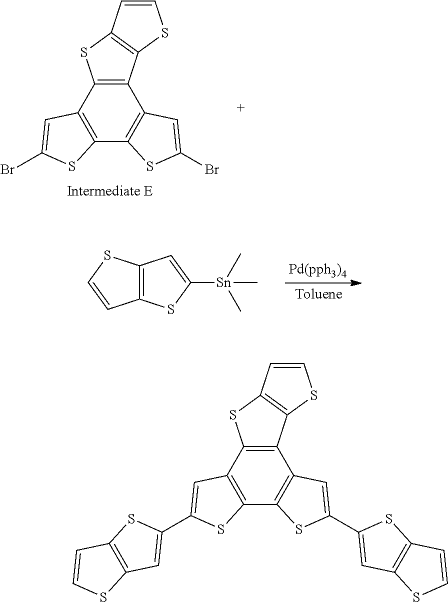 [SMILES: Brc1cc2c(s1)c1sc(Br)cc1c1c3sccc3sc21 ; [CH3][Sn]([CH3])([CH3])[c]1cc2sccc2s1 ; c1cc2sc(-c3cc4c(s3)c3sc(-c5cc6sccc6s5)cc3c3c5sccc5sc43)cc2s1]

A mixture of 2 g (4.4 mmol) of Intermediate E, 2.9 g (9.7 mmol) of trimethyl(thieno[3,2-b]thiophen-2-yl)stannane, 0.5 g (0.44 mmol) of Pd(PPh₃)₄, and 60 ml of toluene was degassed and placed under nitrogen, and then heated at 100° C. for 12 h. After finishing the reaction, the mixture was allowed to cool to room temperature. The organic layer was extracted with dichloromethane and water, dried with anhydrous magnesium sulfate, the solvent was removed and the residue was purified by column chromatography on silica to give product (1.7 g, 66%). MS (m/z, EI+): 578.3.

### Example 2

Synthesis of Compound 2

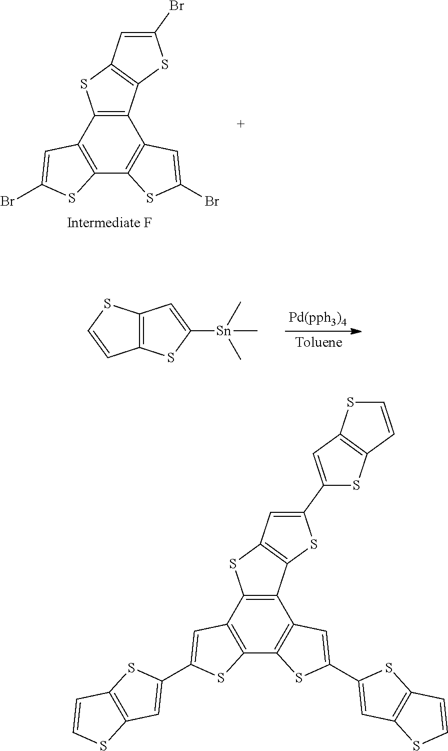 [SMILES: Brc1cc2c(s1)c1sc(Br)cc1c1c3sc(Br)cc3sc21 ; [CH3][Sn]([CH3])([CH3])[c]1cc2sccc2s1 ; c1cc2sc(-c3cc4c(s3)c3sc(-c5cc6sccc6s5)cc3c3c5sc(-c6cc7sccc7s6)cc5sc43)cc2s1]

A mixture of 1 g (1.8 mmol) of Intermediate F, 1.8 g (6.0 mmol) of trimethyl(thieno[3,2-b]thiophen-2-yl)stannane, 0.21 g (0.18 mmol) of Pd(PPh₃)₄, and 30 ml of toluene was degassed and placed under nitrogen, and then heated at 100° C. for 12 h. After finishing the reaction, the mixture was allowed to cool to room temperature. The organic layer was extracted with dichloromethane and water, dried with anhydrous magnesium sulfate, the solvent was removed and the residue was purified by column chromatography on silica to give product (0.7 g, 53%). MS (m/z, EI+): 716.8

### Example 3

Synthesis of Compound 3

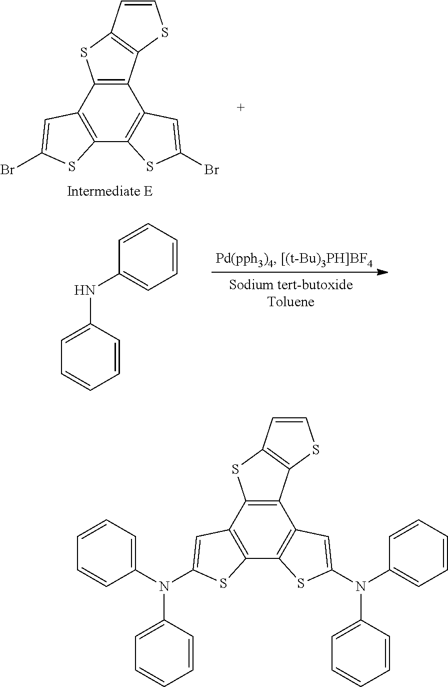 [SMILES: Brc1cc2c(s1)c1sc(Br)cc1c1c3sccc3sc21 ; c1ccc(Nc2ccccc2)cc1 ; c1ccc(N(c2ccccc2)c2cc3c(s2)c2sc(N(c4ccccc4)c4ccccc4)cc2c2c4sccc4sc32)cc1]

A mixture of 2 g (4.4 mmol) of Intermediate E, 1.6 g (9.7 mmol) of diphenylamine, 0.5 g (0.44 mmol) of Pd(PPh₃)₄, 0.06 g (0.22 mmol) Tri-tert-butylphosphoniumtetrafluoroborate, 1.26 g (13.2 mmol) of Sodium tert-butoxide, 60 ml of Toluene was degassed and placed under nitrogen, and then heated at 110° C. for 12 h. After finishing the reaction, the mixture was allowed to cool to room temperature. The organic layer was extracted with dichloromethane and water, dried with anhydrous magnesium sulfate, the solvent was removed and the residue was purified by column chromatography on silica to give product (1.3 g, 48%). MS (m/z, EI+): 637.0

### Example 4

Synthesis of Compound 4

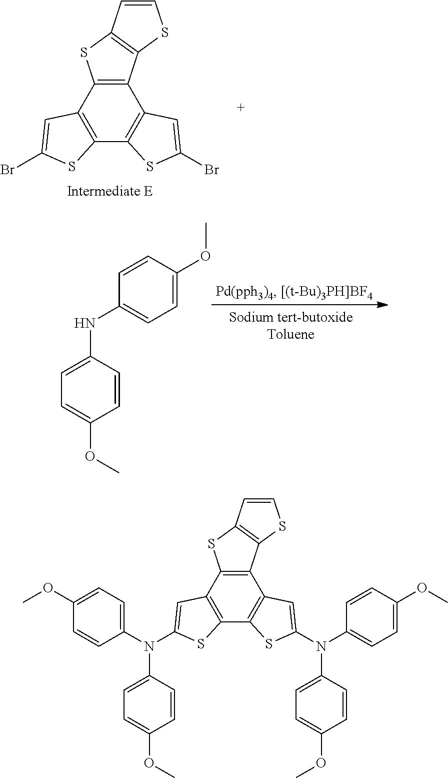

A mixture of 2 g (4.4 mmol) of Intermediate E, 2.2 g (9.7 mmol) of bis(4-methoxyphenyl)amine, 0.5 g (0.44 mmol) of Pd(PPh₃)₄, 0.06 g (0.22 mmol) Tri-tert-butylphosphoniumtetrafluoroborate, 1.26 g (13.2 mmol) of Sodium tert-butoxide, 60 ml of Toluene was degassed and placed under nitrogen, and then heated at 110° C. for 12 h. After finishing the reaction, the mixture was allowed to cool to room temperature. The organic layer was extracted with dichloromethane and water, dried with anhydrous magnesium sulfate, the solvent was removed and the residue was purified by column chromatography on silica to give product (1.3 g, 40%). MS (m/z, EI+): 757.1

### Example 5

Synthesis of Compound 5

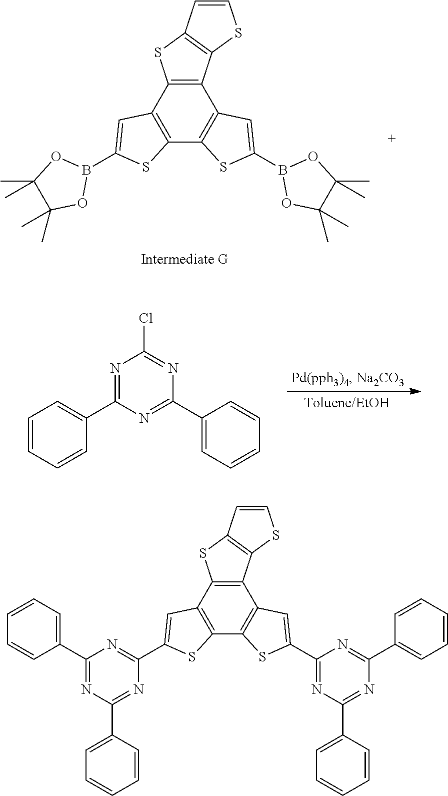

A mixture of 1 g (1.8 mmol) of Intermediate G, 1.44 g (5.4 mmol) of 2-chloro-4,6-diphenyl-1,3,5-triazine, 0.02 g (0.02 mmol) of Pd(PPh₃)₄, 2.7 ml of 2M Na₂CO₃, 20 ml of toluene and 5 ml EtOH was degassed and placed under nitrogen, and then heated at 100° C. for 12 h. After finishing the reaction, the mixture was allowed to cool to room temperature. The organic layer was extracted with dichloromethane and water, dried with anhydrous magnesium sulfate, the solvent was removed and the residue was purified by column chromatography on silica to give product (0.6 g, 45%). MS (m/z, EI+): 764.3

### Example 6

Synthesis of Compound 6

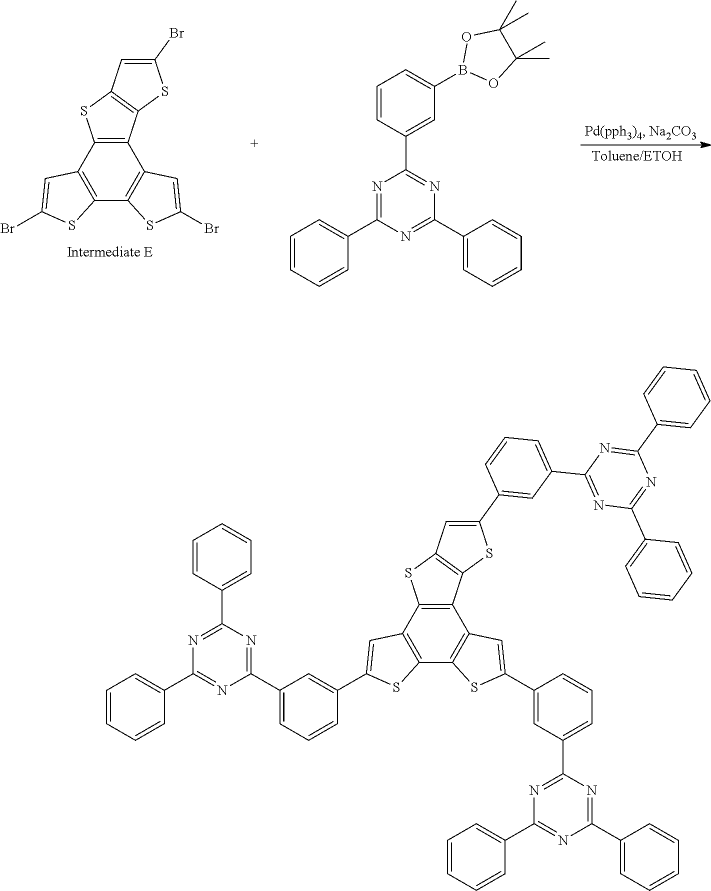 [SMILES: Brc1cc2c(s1)c1sc(Br)cc1c1c3sc(Br)cc3sc21 ; CC1(C)OB(c2cccc(-c3nc(-c4ccccc4)nc(-c4ccccc4)n3)c2)OC1(C)C ; c1ccc(-c2nc(-c3ccccc3)nc(-c3cccc(-c4cc5c(s4)c4sc(-c6cccc(-c7nc(-c8ccccc8)nc(-c8ccccc8)n7)c6)cc4c4c6sc(-c7cccc(-c8nc(-c9ccccc9)nc(-c9ccccc9)n8)c7)cc6sc54)c3)n2)cc1]

A mixture of 2 g (4.4 mmol) of Intermediate F, 6.3 g (14.5 mmol) of 2-chloro-4,6-diphenyl-1,3,5-triazine, 0.05 g (0.04 mmol) of Pd(PPh₃)₄, 8.8 ml of 2M Na₂CO₃, 40 ml of toluene and 10 ml EtOH was degassed and placed under nitrogen, and then heated at 100° C. for 12 h. After finishing the reaction, the mixture was allowed to cool to room temperature. The organic layer was extracted with dichloromethane and water, dried with anhydrous magnesium sulfate, the solvent was removed and the residue was purified by column chromatography on silica to give product (3.5 g, 66%). MS (m/z, FD+): 1223.6

### General Method of Producing Organic Electronic Device

ITO-coated glasses with 9˜12 ohm/square in resistance and 120˜160 nm in thickness are provided (hereinafter ITO substrate) and cleaned in a number of cleaning steps in an ultrasonic bath (e.g. detergent, deionized water). Before vapor deposition of the organic layers, cleaned ITO substrates are further treated by UV and ozone. All pre-treatment processes for ITO substrate are under clean room (class 100).

These organic small molecule layers are applied onto the ITO substrate in order by vapor deposition in a high-vacuum unit (10⁻⁷ Torr), such as: resistively heated quartz boats. The thickness of the respective layer and the vapor deposition rate (0.1˜0.3 nm/sec) are precisely monitored or set with the aid of a quartz-crystal monitor. It is also possible, as described above, for individual layers to consist of more than one compound, i.e. in general a host material doped with a dopant material. This is achieved by co-vaporization from two or more sources.

### Example 7

The substrate of organic thin film transistor (OTFT) device in the present invention is p+-doped Si with thermally grown 250 nm SiO2. The deposition parameters for the sol-gel coated polymethyl methacrylate thin films on the SiO2 gate oxide, its role as a surface modifying layer and the organic semiconcudtor layer deposition procedure are explained elsewhere. Then organic semiconcudtor layer was spin-coated or deposited over the polymethyl methacrylate thin films. Ultrathin LiF layer was then deposited using thermal evaporation onto the organic semiconcudtor layer and the thickness of the LiF layer was varied from 0.1 to 1 nm to get final modified organic semiconcudtor layer. Finally, 60 nm thick aluminum was thermally evaporated onto the modified organic semiconcudtor layer through a shadow mask to form the S/D electrodes. The thickness of the films was monitored by using a quartz crystal monitor. The output characteristics of a device with a channel width of 20 cm and a length of 10 um exhibited typical OTFT characteristics.

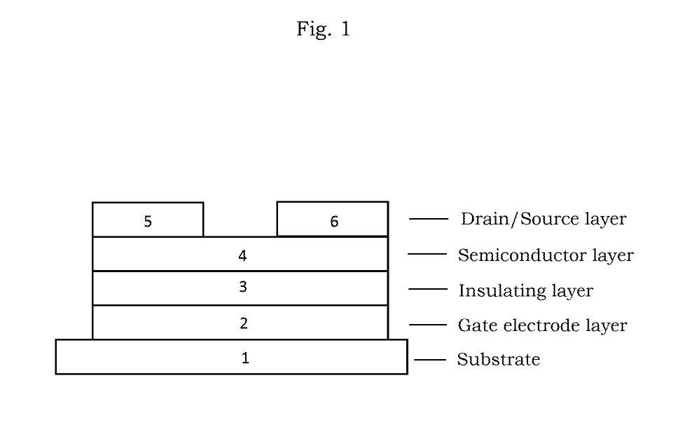

Using a procedure analogous to the above mentioned general method, OTFT device having the following device structures as FIG. 1, organic semiconcudtor layer (EX1˜6), comparable material(Pentacene and 6,13-Bis (tri isopropylsilylethynyl)pentacene (TIPS) were spin-coated or deposited over the device structure to respectively form a thin film.

The electrical measurements of the devices were performed in a nitrogen environment inside a glove box using HP 4156C and Keithley 4200 semiconductor parameter analyzer. The capacitance-voltage (C-V) measurement was performed by Agilent E4980A precision LCR meter.

The prior art of OTFT materials for producing standard OTFT device control and comparable material in this invention shown its chemical structure as follows:

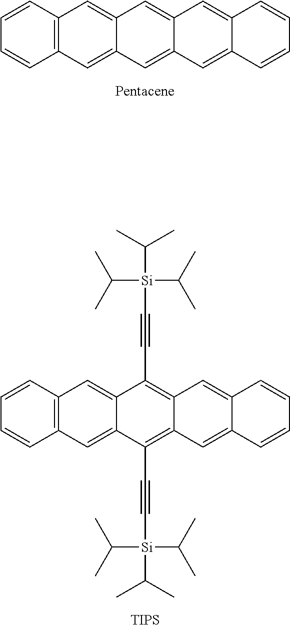

The field-effect carrier mobility and on/off current ratio of OTFT device data are shown as Table 1.

TABLE 1

| Semiconductors | Field-effect mobility | On/Off |
|---|---|---|
| Example | (cm²V⁻¹s⁻¹) | current ratio |
| Ex1 | 5.0 × 10⁰ | 3.0 × 10⁴ |
| Ex2 | 3.5 × 10⁰ | 2.5 × 10⁴ |
| Ex3 | 7.0 × 10⁻² | 4.4 × 10⁵ |
| Ex4 | 1.5 × 10⁻² | 3.2 × 10⁶ |
| Ex5 | 7.8 × 10⁻¹ | 7.7 × 10⁵ |
| Ex6 | 2.3 × 10⁻¹ | 2.0 × 10⁵ |
| Comparative 1 | 3.5 × 10⁻¹ | 7.5 × 10⁴ |
| (Pentacene) | | |
| Comparative 2 | 5.0 × 10⁻² | 9.0 × 10⁴ |
| (TIPS) | | |

In the above preferred embodiments for OTFT device test report (see Table 1), we show that the heterocyclic compound with a general formula (I) in the present invention used as organic thin-film material for OTFT device display good performance shown the OTFT exhibited an on/off current ratio.

### Example 8

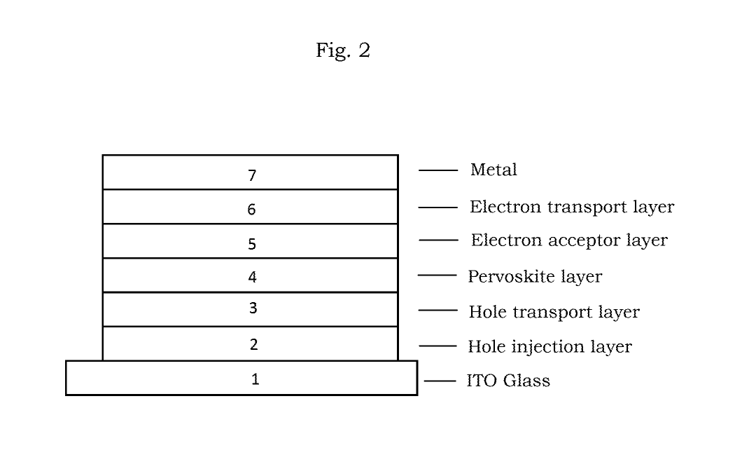

Using a procedure analogous to the above mentioned general method, the perovskite solar cell device having the following device structure was produced (See FIG. 2). ITO/PEDOT:PSS/Ex1˜Ex6(30 nm)/Pervoskite layer: CH₃NH₃PbI₃/PCBM/BCP(100 nm)/Al(100 nm).

Hole injection layer (HI): PEDOT:PSS(AI4083) was spin-coated (4000 rpm) onto the ITO surfaces for 1 min, followed by annealing at 130° C. for 30 min.

HT layer: EX1˜EX6 were deposited through thermal evaporation.

Pervoskite layer: the PbI₂(40 wt %)(99%, Alfa Aesar) were dissolved in anhydrous dimethyl sulfoxide (DMSO) and stirred on a hot plate at 70° C. overnight. The hot solution of PbI₂ was spin coated onto the PEDOT:PSS film at 4000 rpm (40 sec) and the sample was kept on the hot plate at 70° C. for 30 min.

The CH₃NH₃PbI₃(2 wt %) were dissolved in anhydrous 2-proponal and stirred on a hot plate at 70° C. overnight. The hot solution of CH₃NH₃PbI₃ was spin coated onto the PbI₂ film at 5000 rpm (40 sec) and the sample was kept on the hot plate at 100° C. for 120 min.

Electron acceptor layer (ET): a solution (20 mg/mL) of [6,6]-phenyl-C₆₁-butyric acid methyl ester (PC₆₁BM)(85 nm) in dichlorobenzene (CB) was spun (6000 rpm, 60 s) onto the perovskite layer, followed by annealing at 90° C. for 30 min.

Electron transport layer (ET) & Cathode: The device structure was completed through sequential thermal evaporation of 2,9-dimethyl-4,7-diphenyl-1,10-phenanthroline (BCP)(10 nm), and an aluminum electrode (100 nm) through a shadow mask under vacuum.

[6,6]-phenyl-C₆₁-butyric acid methyl ester (PC₆₁BM) use for electron acceptor material. 2,9-dimethyl-4,7-diphenyl-1,10-phenanthroline (BCP) used as electron transport material. The prior art of organic materials for producing standard pervoskite device control HT compounds (EX1˜6) and comparable with Poly[N,N′-bis(4-butylphenyl)-N,N′-bis(phenyl)-benzidine](Poly-TPD)(Comparative 1) in this invention shown its chemical structure as follows:

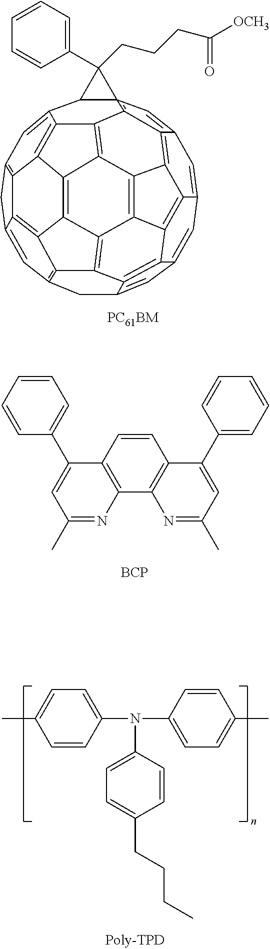

A solar simulator was used to irradiate a perovskite solar cell with light at an illuminance of 100 mW/cm²(1 SUN). After the current-voltage characteristic stabilized, the current-voltage characteristic was measured and the conversion efficiency was determined as the initial conversion efficiency. The ratio of the conversion efficiency after the heating test to the initial conversion efficiency was calculated as a retention ratio.

The I-V data (at 1 SUN) of pervoskite device testing report as Table2.

TABLE 2

| | | | Power Conversion |
|---|---|---|---|
| | Open | Current Density | Efficiency |
| HT Example | Voltage (Voc) | (mA/cm2) | (%) |
| Ex1 | 0.97 | 11.6 | 7.1% |
| Ex2 | 0.97 | 8.8 | 6.5% |
| Ex3 | 0.98 | 19.3 | 12.1% |
| Ex4 | 0.97 | 18.8 | 11.9% |
| Ex5 | 0.95 | 9.6 | 6.4% |
| Ex6 | 0.94 | 10.8 | 5.8% |
| Comparative 1 | 0.98 | 17.6 | 9.8% |

In the above preferred embodiments for pervoskite solar device test report (see Table2), we show that the with a general formula(I) in the present invention display good performance than the prior art of pervoskite solar cell for hole transport materials.

The present invention discloses a heterocyclic compound represented by the formula (I) as the follows, the heterocyclic compound is suitable for an organic semiconductor device, a pervoskite solar cell device, and organic electroluminescence (organic EL) device. Additional, the present invention employs the heterocyclic compound as hole transport layer (HTL) or electron transport layer (ETL) materials for pervoskite solar cell device and organic EL device can display excellent performance.

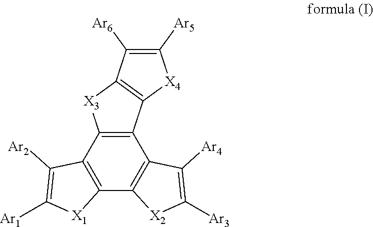

wherein X₁ to X₄ each independently represent a sulfur or a selenium atom, Ar₁ to Ar₆ are identical or different, Ar₁ to Ar₆ are independently selected from the group consisting of a hydrogen atom, a halide, —CN, —NC, —NCS, —SCN, —NH₂, —OH, —NO₂, —CF₃, —NC, a substituted or unsubstituted alkyl group having 1 to 30 carbon atoms, a substituted or unsubstituted aryl group having 6 to 30 carbon atoms, a substituted or unsubstituted heteroaryl group having 3 to 30 carbon atoms, a substituted or unsubstituted aralkyl group having 6 to 30 carbon atoms, a substituted or unsubstituted alkoxyl group having 6 to 30 carbon atoms, a substituted or unsubstituted alkenyl group having 6 to 30 carbon atoms and a substituted or unsubstituted alkylamine group having 6 to 30 carbon atoms.

1. A heterocyclic compound represented by the formula (I) as follows: 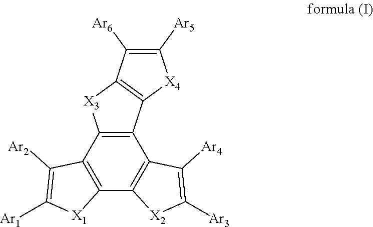
wherein X₁ to X₄ each independently represent a sulfur or a selenium atom, Ar₁ to Ar₆ are identical or different, Ar₁ to Ar₆ are independently selected from the group consisting of a hydrogen atom, a halide, —CN, —NC, —NCS, —SCN, —NH₂, —OH, —NO₂, —CF₃, —NC, a substituted or unsubstituted alkyl group having 1 to 30 carbon atoms, a substituted or unsubstituted aryl group having 6 to 30 carbon atoms, a substituted or unsubstituted heteroaryl group having 3 to 30 carbon atoms, a substituted or unsubstituted aralkyl group having 6 to 30 carbon atoms, a substituted or unsubstituted alkoxyl group having 6 to 30 carbon atoms, a substituted or unsubstituted alkenyl group having 6 to 30 carbon atoms and a substituted or unsubstituted arylamine group having 6 to 30 carbon atoms, with the proviso that when all of X₁ to X₄ represent a sulfur atom, at least one of Ar₁ to Ar₆ is not a hydrogen atom.

2. The heterocyclic compound according to claim 1, wherein Ar₁ to Ar₆ are selected from the group consisting of: 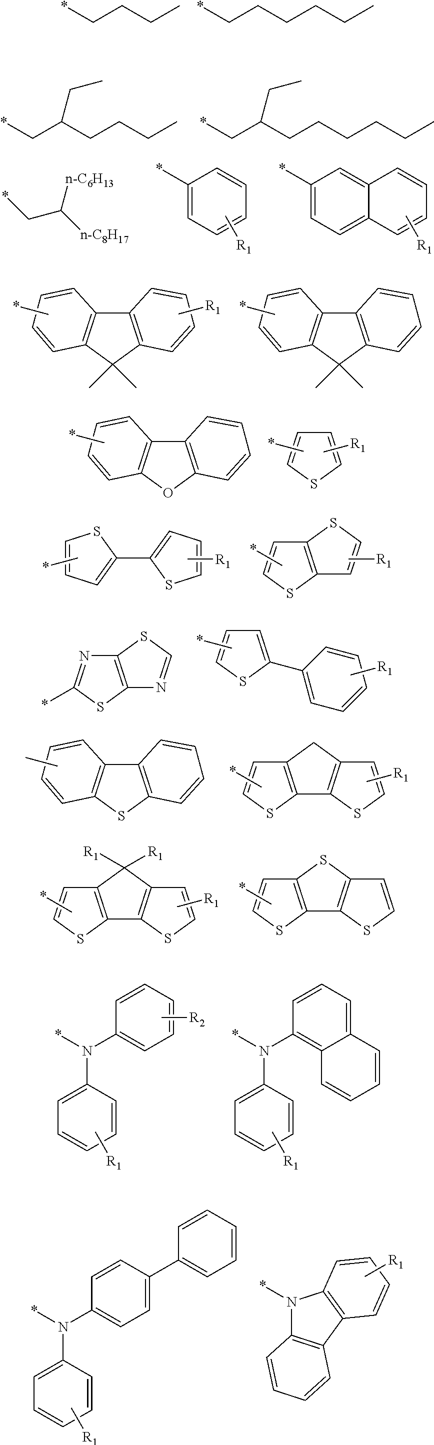 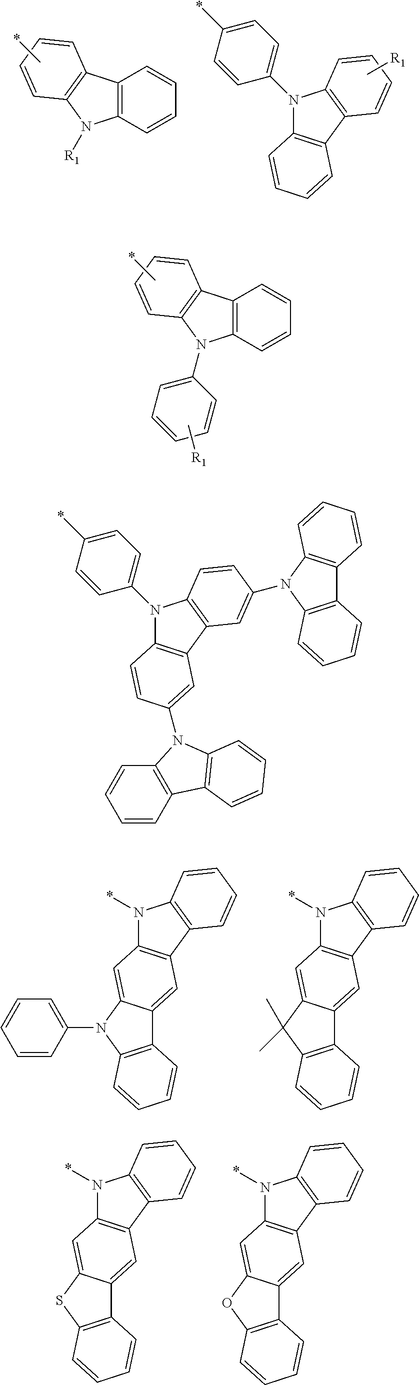 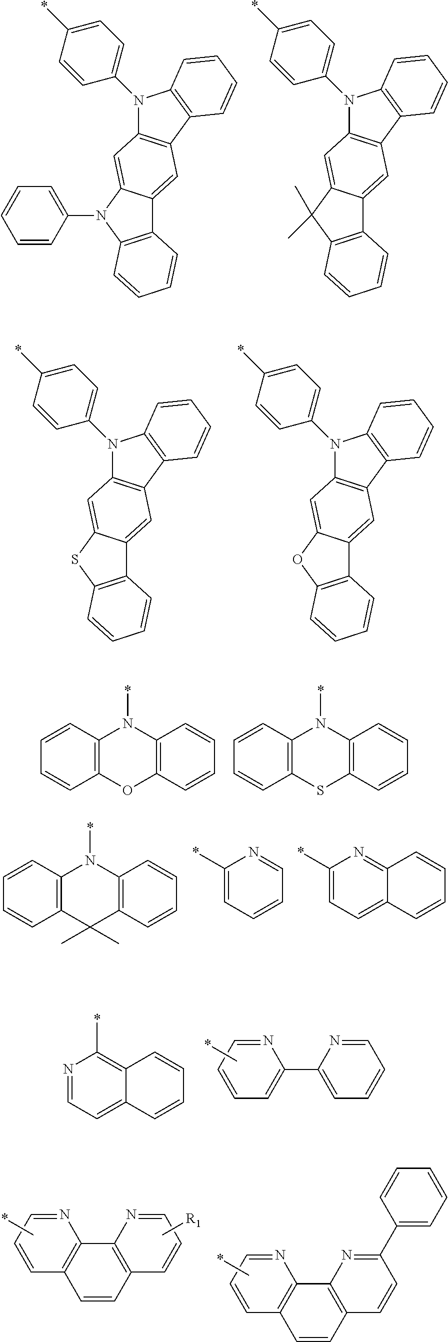 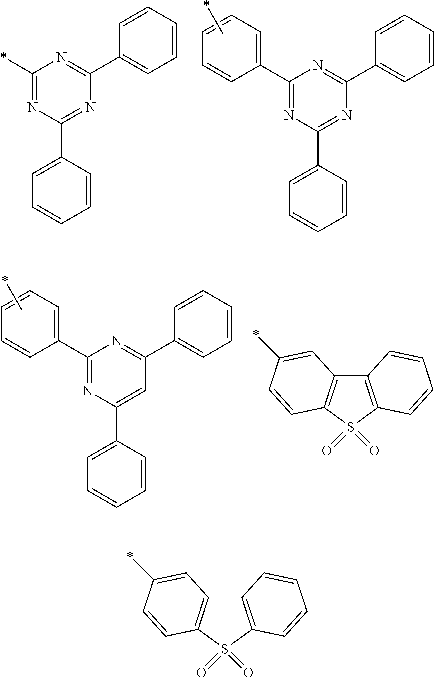
wherein R₁ and R₂ represent alkyl group, or aryl group.

3. The heterocyclic compound according to claim 1, wherein the heterocyclic compound is selected from the group consisting of:

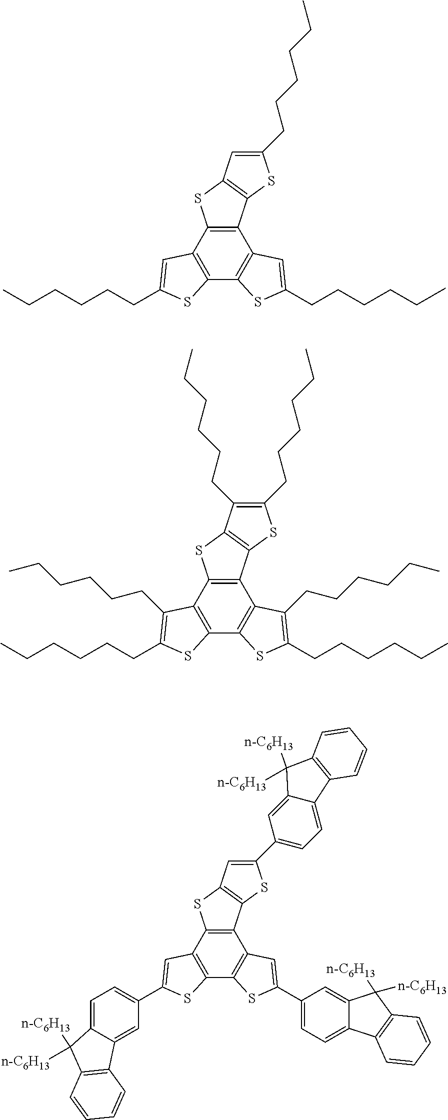

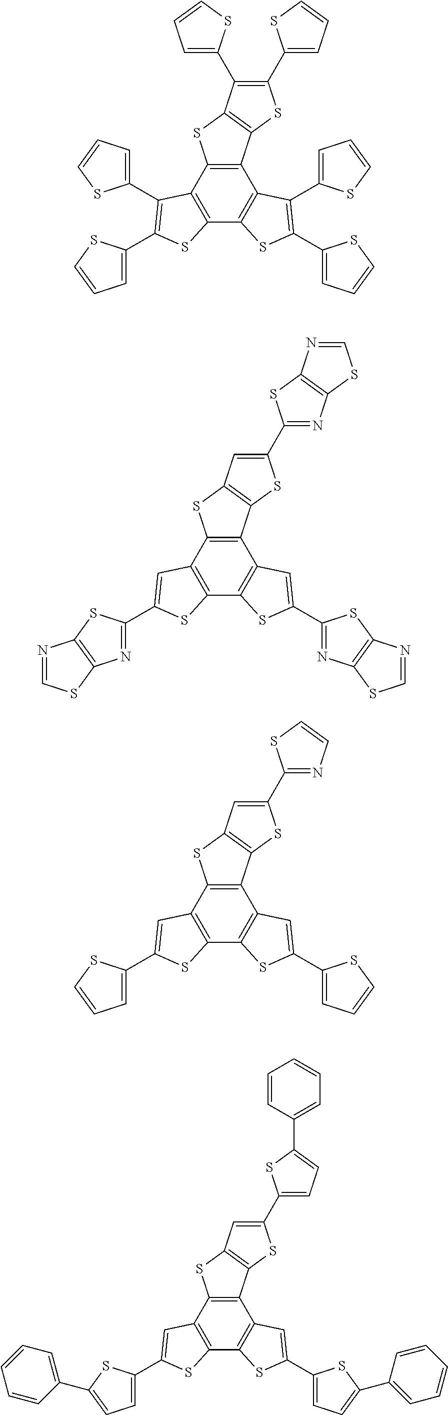 [SMILES: c1csc(-c2sc3c(sc4c5c(-c6cccs6)c(-c6cccs6)sc5c5sc(-c6cccs6)c(-c6cccs6)c5c34)c2-c2cccs2)c1 ; c1nc2sc(-c3cc4c(s3)c3sc(-c5nc6scnc6s5)cc3c3c5sc(-c6nc7scnc7s6)cc5sc43)nc2s1 ; c1csc(-c2cc3c(s2)c2sc(-c4cccs4)cc2c2c4sc(-c5nccs5)cc4sc32)c1 ; c1ccc(-c2ccc(-c3cc4c(s3)c3sc(-c5ccc(-c6ccccc6)s5)cc3c3c5sc(-c6ccc(-c7ccccc7)s6)cc5sc43)s2)cc1]

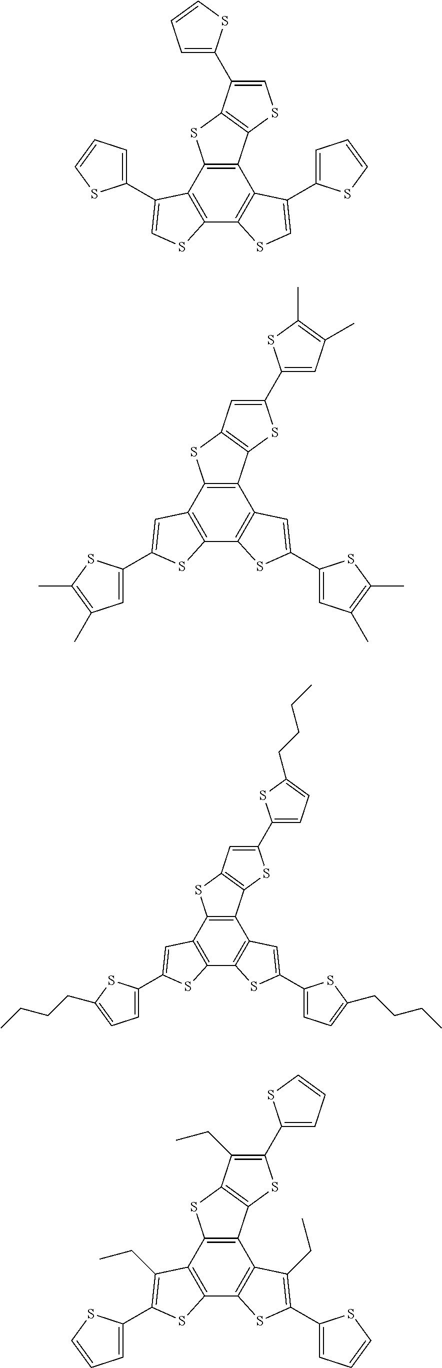

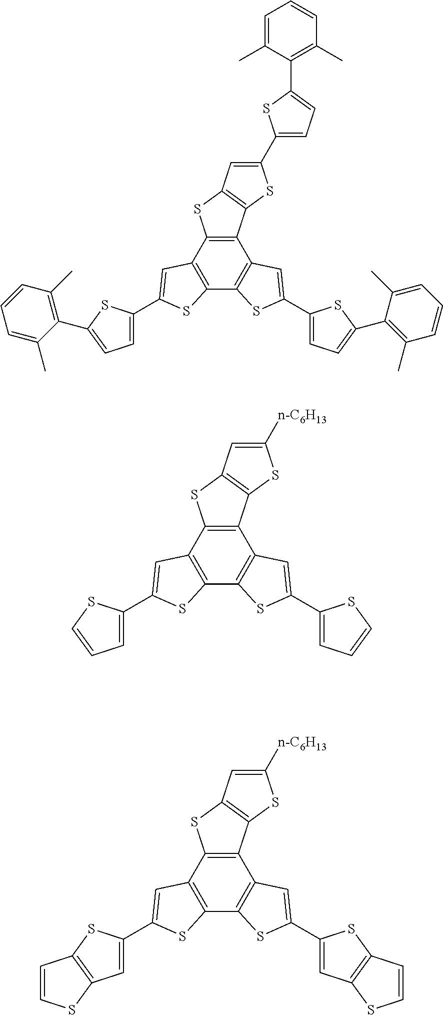

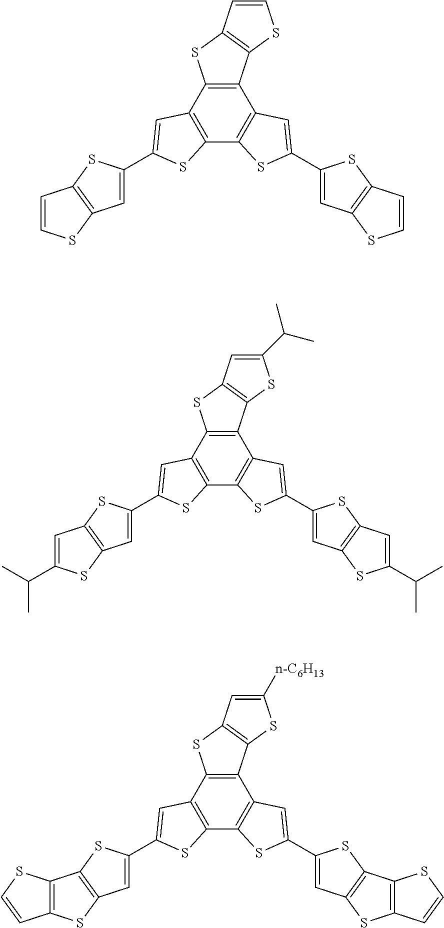

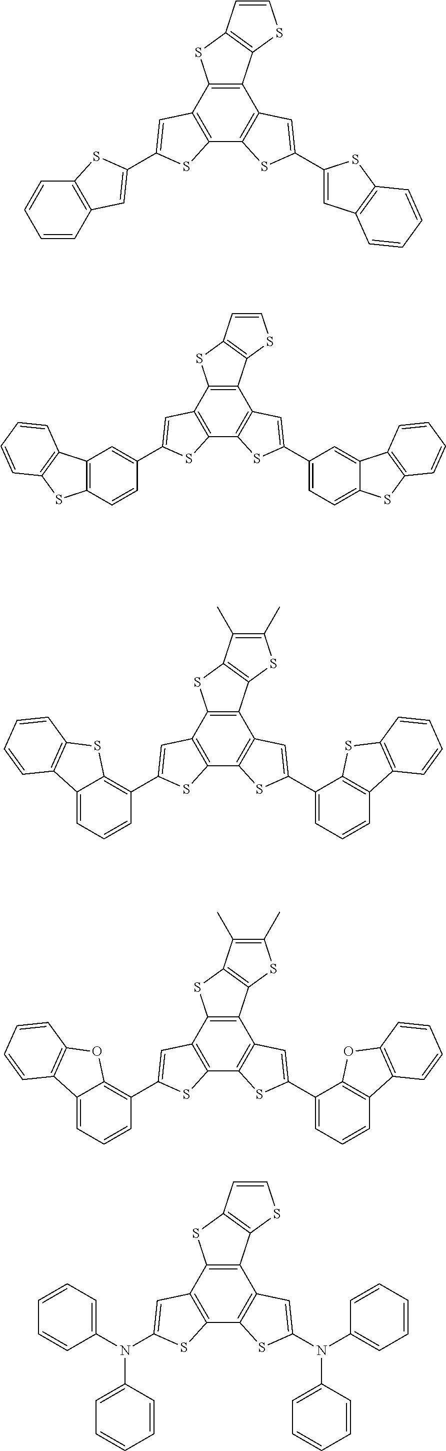 [SMILES: c1ccc2sc(-c3cc4c(s3)c3sc(-c5cc6ccccc6s5)cc3c3c5sccc5sc43)cc2c1 ; c1ccc2c(c1)sc1ccc(-c3cc4c(s3)c3sc(-c5ccc6sc7ccccc7c6c5)cc3c3c5sccc5sc43)cc12 ; Cc1sc2c(sc3c4cc(-c5cccc6c5sc5ccccc56)sc4c4sc(-c5cccc6c5sc5ccccc56)cc4c23)c1C ; Cc1sc2c(sc3c4cc(-c5cccc6c5oc5ccccc56)sc4c4sc(-c5cccc6c5oc5ccccc56)cc4c23)c1C ; c1ccc(N(c2ccccc2)c2cc3c(s2)c2sc(N(c4ccccc4)c4ccccc4)cc2c2c4sccc4sc32)cc1]

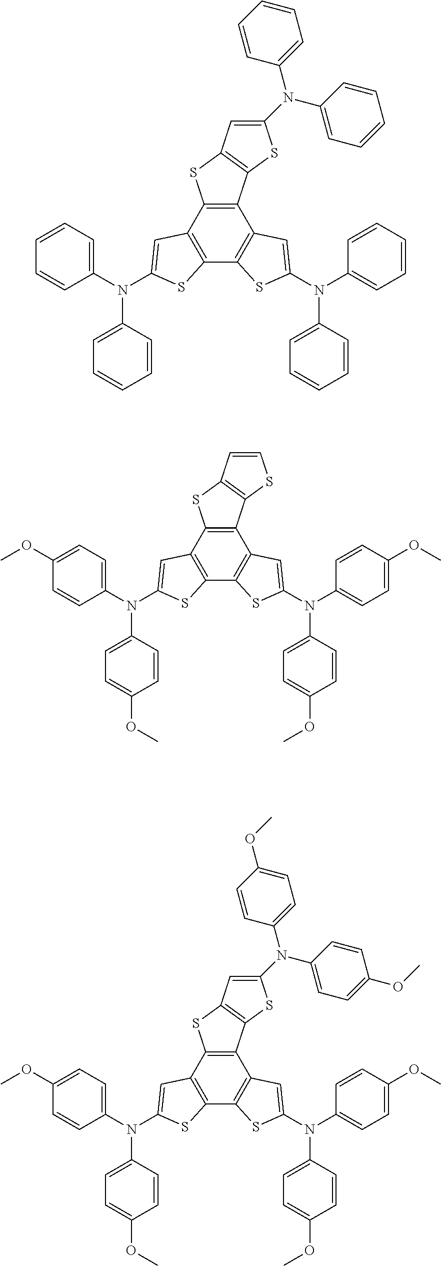

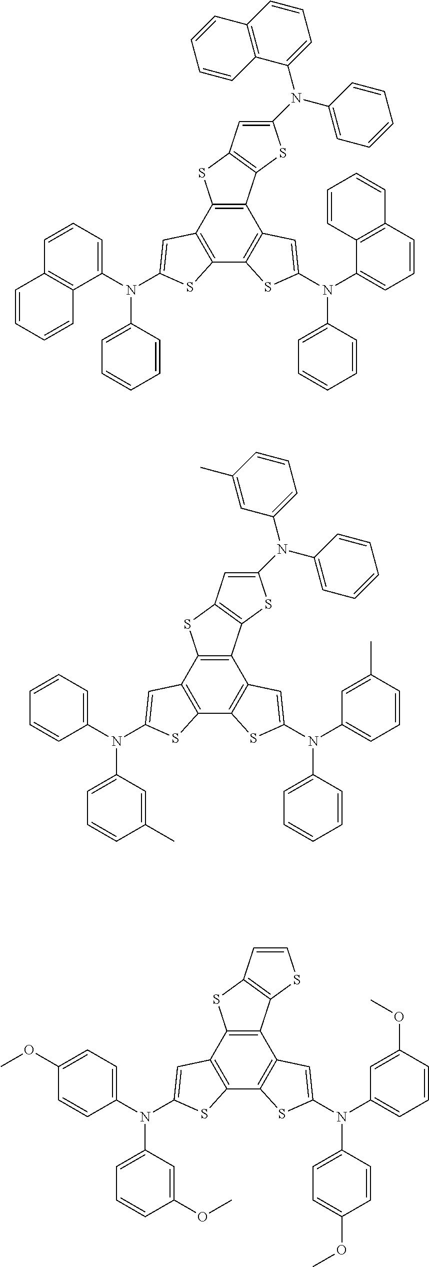 [SMILES: c1ccc(N(c2cc3c(s2)c2sc(N(c4ccccc4)c4cccc5ccccc45)cc2c2c4sc(N(c5ccccc5)c5cccc6ccccc56)cc4sc32)c2cccc3ccccc23)cc1 ; Cc1cccc(N(c2ccccc2)c2cc3c(s2)c2sc(N(c4ccccc4)c4cccc(C)c4)cc2c2c4sc(N(c5ccccc5)c5cccc(C)c5)cc4sc32)c1 ; COc1ccc(N(c2cccc(OC)c2)c2cc3c(s2)c2sc(N(c4ccc(OC)cc4)c4cccc(OC)c4)cc2c2c4sccc4sc32)cc1]

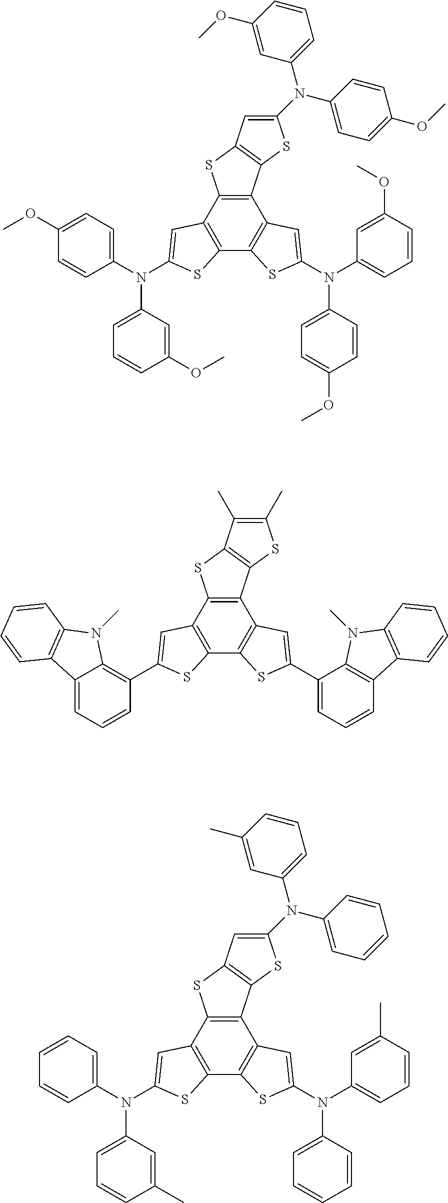

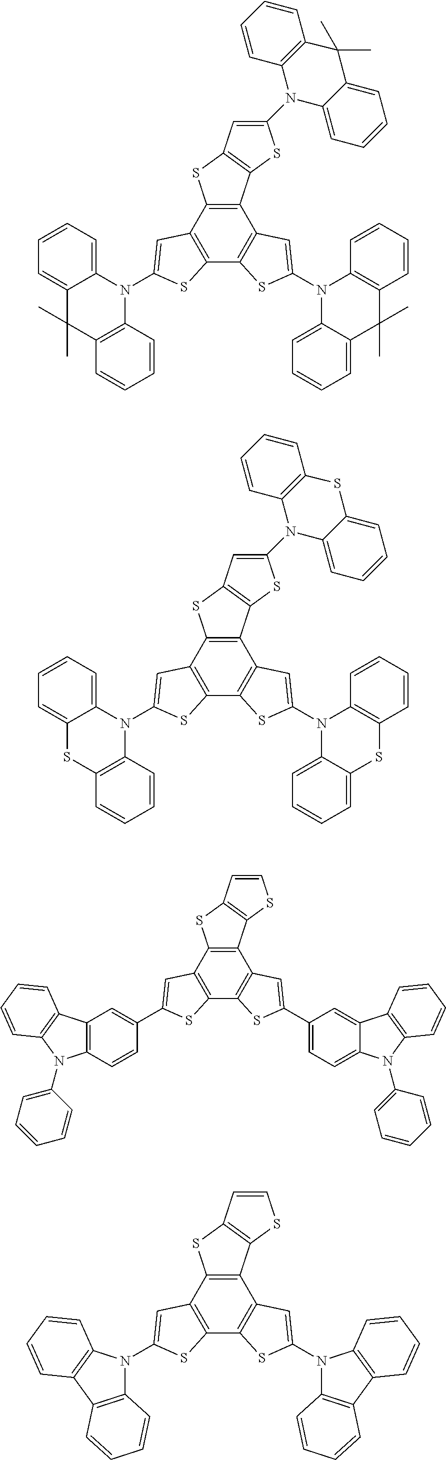 [SMILES: CC1(C)c2ccccc2N(c2cc3c(s2)c2sc(N4c5ccccc5C(C)(C)c5ccccc54)cc2c2c4sc(N5c6ccccc6C(C)(C)c6ccccc65)cc4sc32)c2ccccc21 ; c1ccc2c(c1)Sc1ccccc1N2c1cc2c(s1)c1sc(N3c4ccccc4Sc4ccccc43)cc1c1c3sc(N4c5ccccc5Sc5ccccc54)cc3sc21 ; c1ccc(-n2c3ccccc3c3cc(-c4cc5c(s4)c4sc(-c6ccc7c(c6)c6ccccc6n7-c6ccccc6)cc4c4c6sccc6sc54)ccc32)cc1 ; c1ccc2c(c1)c1ccccc1n2-c1cc2c(s1)c1sc(-n3c4ccccc4c4ccccc43)cc1c1c3sccc3sc21]

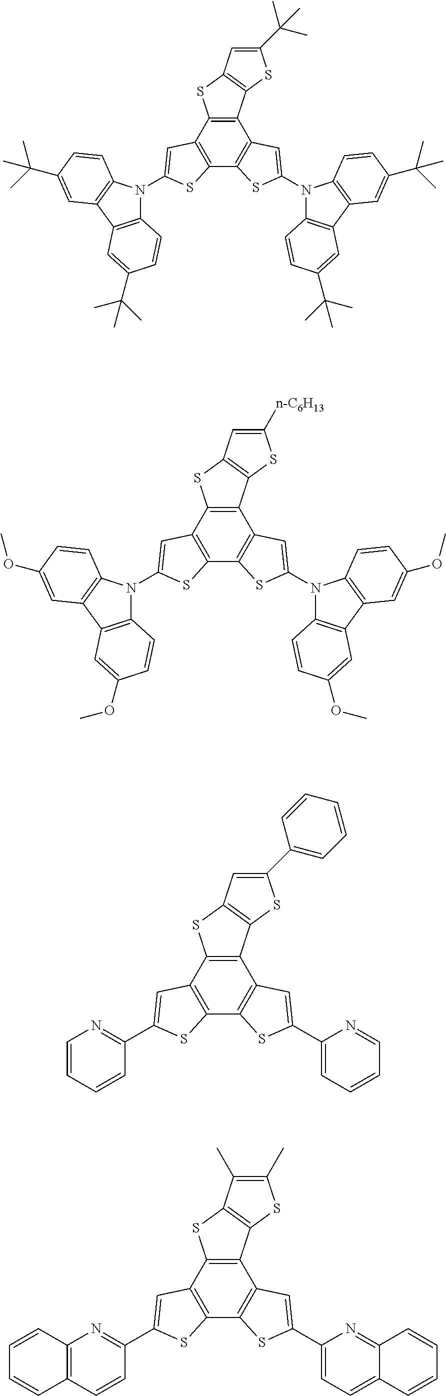

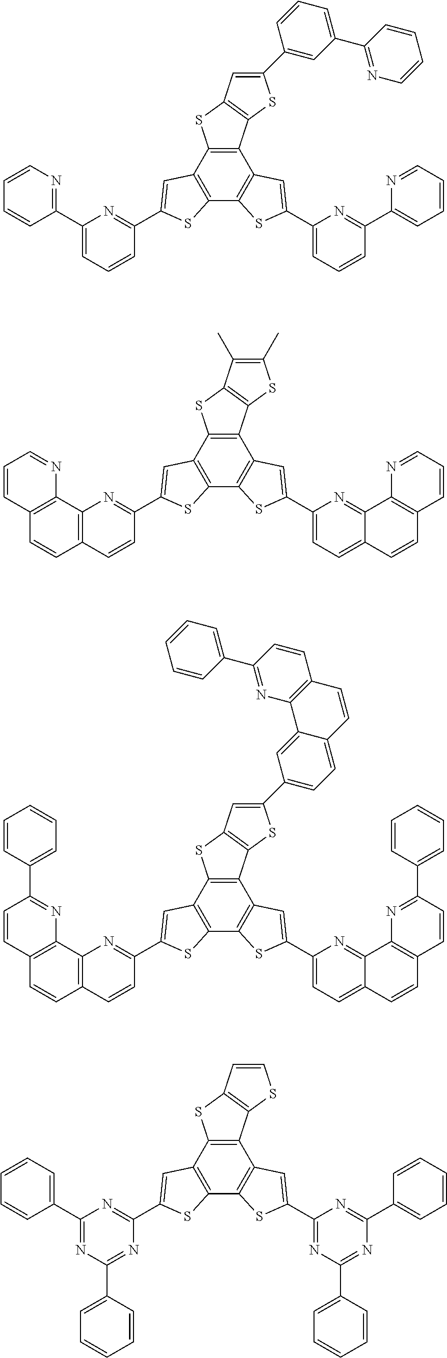 [SMILES: c1ccc(-c2cccc(-c3cc4sc5c6cc(-c7cccc(-c8ccccn8)n7)sc6c6sc(-c7cccc(-c8ccccn8)n7)cc6c5c4s3)c2)nc1 ; Cc1sc2c(sc3c4cc(-c5ccc6ccc7cccnc7c6n5)sc4c4sc(-c5ccc6ccc7cccnc7c6n5)cc4c23)c1C ; c1ccc(-c2ccc3ccc4ccc(-c5cc6sc7c8cc(-c9ccc%10ccc%11ccc(-c%12ccccc%12)nc%11c%10n9)sc8c8sc(-c9ccc%10ccc%11ccc(-c%12ccccc%12)nc%11c%10n9)cc8c7c6s5)cc4c3n2)cc1 ; c1ccc(-c2nc(-c3ccccc3)nc(-c3cc4c(s3)c3sc(-c5nc(-c6ccccc6)nc(-c6ccccc6)n5)cc3c3c5sccc5sc43)n2)cc1]

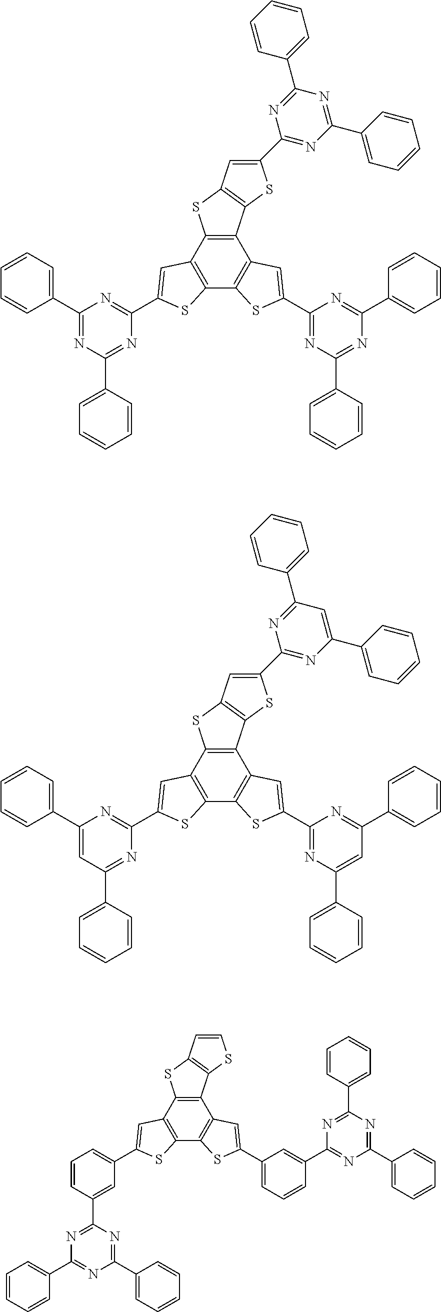 [SMILES: c1ccc(-c2nc(-c3ccccc3)nc(-c3cc4c(s3)c3sc(-c5nc(-c6ccccc6)nc(-c6ccccc6)n5)cc3c3c5sc(-c6nc(-c7ccccc7)nc(-c7ccccc7)n6)cc5sc43)n2)cc1 ; c1ccc(-c2cc(-c3ccccc3)nc(-c3cc4c(s3)c3sc(-c5nc(-c6ccccc6)cc(-c6ccccc6)n5)cc3c3c5sc(-c6nc(-c7ccccc7)cc(-c7ccccc7)n6)cc5sc43)n2)cc1 ; c1ccc(-c2nc(-c3ccccc3)nc(-c3cccc(-c4cc5c(s4)c4sc(-c6cccc(-c7nc(-c8ccccc8)nc(-c8ccccc8)n7)c6)cc4c4c6sccc6sc54)c3)n2)cc1]

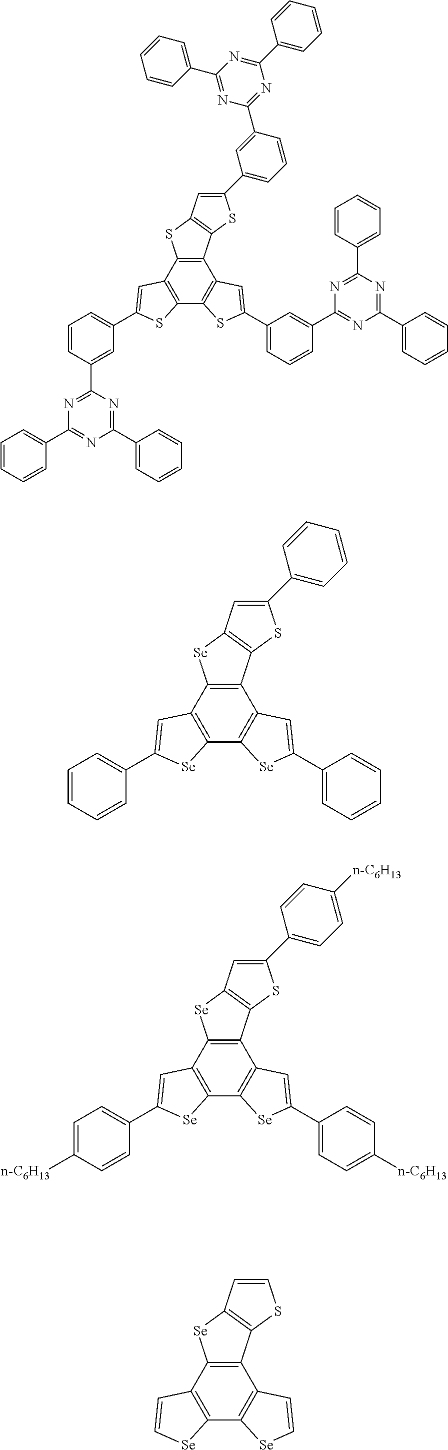

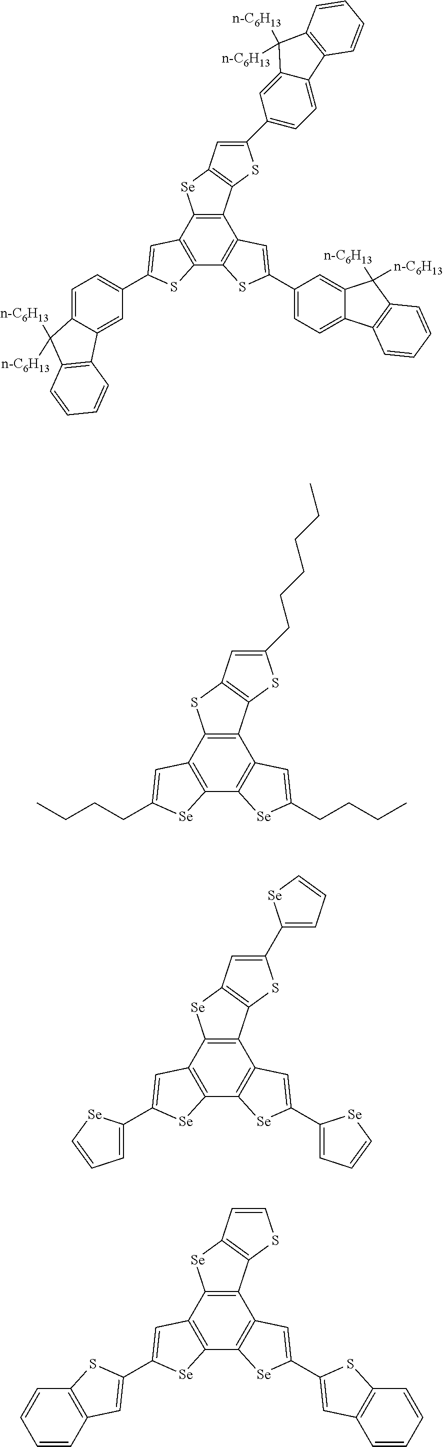

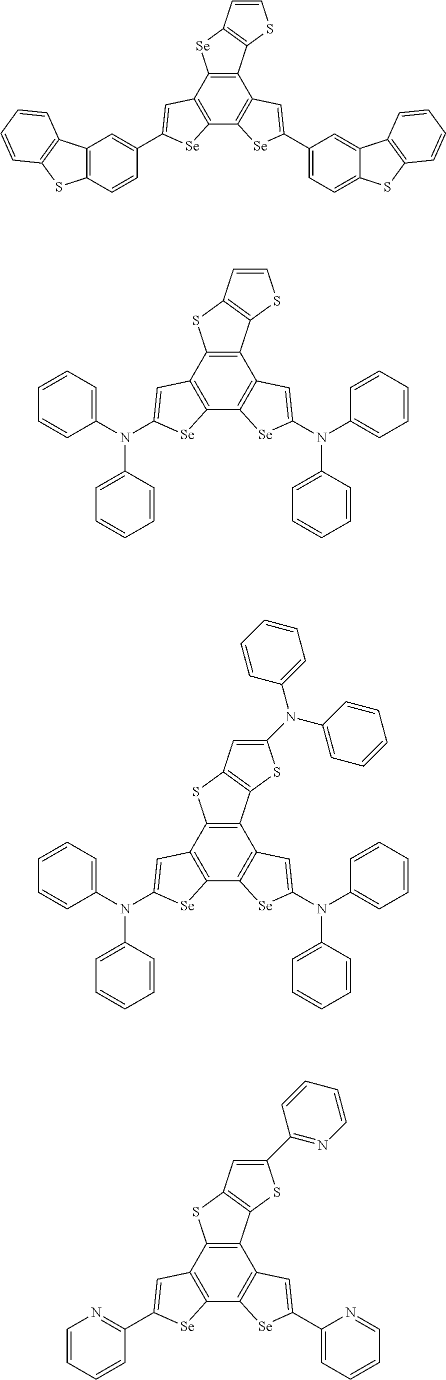 [SMILES: c1ccc2c(c1)sc1ccc(-c3cc4c([se]3)c3[se]c(-c5ccc6sc7ccccc7c6c5)cc3c3c5sccc5[se]c43)cc12 ; c1ccc(N(c2ccccc2)c2cc3c4sc5ccsc5c4c4cc(N(c5ccccc5)c5ccccc5)[se]c4c3[se]2)cc1 ; c1ccc(N(c2ccccc2)c2cc3sc4c5cc(N(c6ccccc6)c6ccccc6)[se]c5c5[se]c(N(c6ccccc6)c6ccccc6)cc5c4c3s2)cc1 ; c1ccc(-c2cc3sc4c5cc(-c6ccccn6)[se]c5c5[se]c(-c6ccccn6)cc5c4c3s2)nc1]

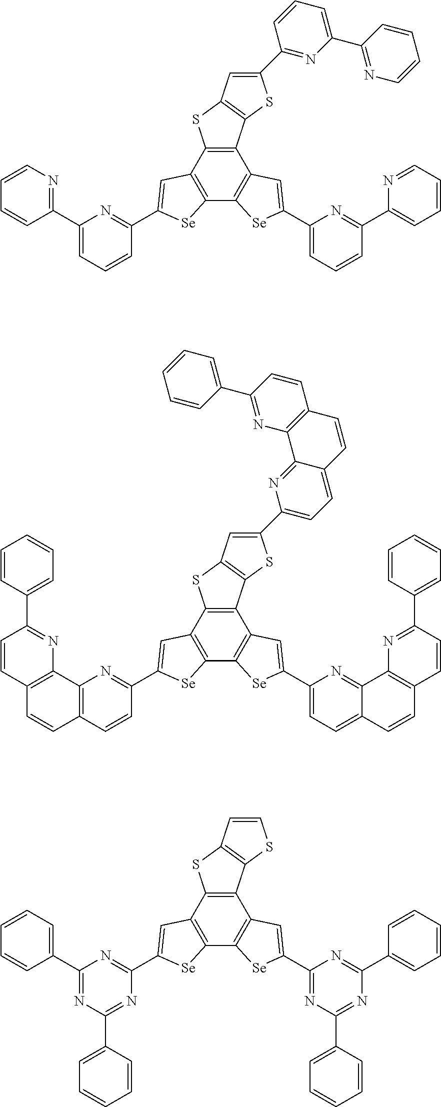 [SMILES: c1ccc(-c2cccc(-c3cc4sc5c6cc(-c7cccc(-c8ccccn8)n7)[se]c6c6[se]c(-c7cccc(-c8ccccn8)n7)cc6c5c4s3)n2)nc1 ; c1ccc(-c2ccc3ccc4ccc(-c5cc6sc7c8cc(-c9ccc%10ccc%11ccc(-c%12ccccc%12)nc%11c%10n9)[se]c8c8[se]c(-c9ccc%10ccc%11ccc(-c%12ccccc%12)nc%11c%10n9)cc8c7c6s5)nc4c3n2)cc1 ; c1ccc(-c2nc(-c3ccccc3)nc(-c3cc4c5sc6ccsc6c5c5cc(-c6nc(-c7ccccc7)nc(-c7ccccc7)n6)[se]c5c4[se]3)n2)cc1]

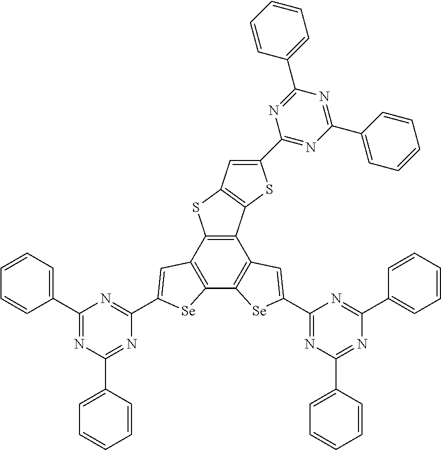 [SMILES: c1ccc(-c2nc(-c3ccccc3)nc(-c3cc4sc5c6cc(-c7nc(-c8ccccc8)nc(-c8ccccc8)n7)[se]c6c6[se]c(-c7nc(-c8ccccc8)nc(-c8ccccc8)n7)cc6c5c4s3)n2)cc1]

4. An organic electronic device comprising the heterocyclic compound with formula(I) according to claim 1.

5. The organic electronics device according to claim 4, wherein the device is organic semiconductor device, pervoskite solar cell device, and organic electroluminescence (organic EL) device.

6. An organic semiconductor device comprising a gate electrode, a metal oxide layer, an adhesive layer, a drain electrode, a source electrode, and an active layer.

7. The organic semiconductor device according to claim 6, wherein the active layer comprising the heterocyclic compound with formula(I) according to claim 1.

8. The organic semiconductor device according to claim 6, wherein the gate electrode is silicon, doped silicon or aluminum.

9. The organic semiconductor device according to claim 6, wherein the metal oxide layer is silicon oxide or aluminum oxide.

10. The organic semiconductor device according to claim 6, wherein adhesive layer is titanium, tungsten, or chromium.

11. The organic semiconductor device according to claim 6, wherein the drain electrode is gold or platinum.

12. The organic semiconductor device according to claim 6, wherein the source electrode comprising is a layer of gold or a layer of platinum.

13. The organic EL device, wherein the hole transport layer or hole injection layer comprising the heterocyclic compound with formula(I) according to claim 1.

14. The organic EL device, wherein the electron transport layer comprising the heterocyclic compound with formula(I) according to claim 1.

15. The pervoskite solar cell device, wherein the hole transport layer comprising the heterocyclic compound with formula(I) according to claim 1.

16. The pervoskite solar cell device, wherein the electron transport layer comprising the heterocyclic compound with formula(I) according to claim 1.

17. The organic solar cell device, wherein the hole transport layer comprising the heterocyclic compound with formula(I) according to claim 1.

18. The organic solar cell device, wherein the electron transport layer comprising the heterocyclic compound with formula(I) according to claim 1.
